SEQUENCE LISTING (22 sequences)

SEQ ID NO: 1 - protein, 116 aa, synthetic construct
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLRYSSGWGQGTMVTVSS

SEQ ID NO: 2 - protein, 110 aa, synthetic construct
QPVLTQPPSVSKGLRQTATLTCSGNSNNVGNAGAVWLQQHQGHPPKLLSYRNNNRPSGISERISASRSGNTASLTISGLQPEDEADYYCSAWDSSLRVQVFGGGTKLTVL

SEQ ID NO: 3 - protein, 120 aa, synthetic construct
QVTLQQSGPGLVKPSQTLSLTCAISGDSVSSNSAAWNWIRQSPSRGLEWLGRTYYRSKWFTYYAASVKGRISINRDTSKNQFSLQLNSVTPDDTAVYFCARGSGGYIDHWGQGTLVTVSS

SEQ ID NO: 4 - protein, 115 aa, synthetic construct
QAVLTQPSSLSASPGASASLTCTLRSGINVDSYRIYWYQQKPGSPPQYLLRYKSDSDKQQGSGVPSRFSGSKDASANAGILLISGLQSEDEADYYCMIWHSSAVVFGGGTKLTVL

SEQ ID NO: 5 - protein, 10 aa, synthetic construct
GFTFSSYAMS

SEQ ID NO: 6 - protein, 11 aa, synthetic construct
SAISGSGGSTY

SEQ ID NO: 7 - protein, 7 aa, synthetic construct
GLRYSSG

SEQ ID NO: 8 - protein, 13 aa, synthetic construct
SGNSNNVGNAGAV

SEQ ID NO: 9 - protein, 6 aa, synthetic construct
NNNRPS

SEQ ID NO: 10 - protein, 11 aa, synthetic construct
SAWDSSLRVQV

SEQ ID NO: 11 - protein, 12 aa, synthetic construct
GDSVSSNSAAWN

SEQ ID NO: 12 - protein, 12 aa, synthetic construct
GRTYYRSKWFTY

SEQ ID NO: 13 - protein, 8 aa, synthetic construct
GSGGYIDH

SEQ ID NO: 14 - protein, 14 aa, synthetic construct
TLRSGINVDSYRIY

SEQ ID NO: 15 - protein, 6 aa, synthetic construct
DKQQGS

SEQ ID NO: 16 - protein, 9 aa, synthetic construct
MIWHSSAVV

SEQ ID NO: 17 - protein, 15 aa, synthetic construct
GGGGSGGGGSGGGGS

SEQ ID NO: 18 - protein, 10 aa, synthetic construct
VVVGACGVGK

SEQ ID NO: 19 - DNA, 15 bp, Homo sapiens
  source 1..15: mol_type = genomic DNA
catttcccgtaaatc

SEQ ID NO: 20 - protein, 5 aa, synthetic construct
SSSSG

SEQ ID NO: 21 - protein, 15 aa, synthetic construct
SSSSGSSSSGSSSSG

SEQ ID NO: 22 - protein, 5 aa, synthetic construct
GGGGS

(19) United States
(12) Patent Application Publication
(10) Pub. No.: US 2026/0234290 A1
(43) Pub. Date: Aug. 13, 2026

(54) Antibodies to Stat3 Decoy Oligonucleotide

(71) Applicant: The Regents of the University of California, Oakland, CA (US)

(72) Inventors: Charles S. Craik, San Francisco, CA (US); Dong hee Chung, San Francisco, CA (US)

(21) Appl. No.: 19/486,839

(22) PCT Filed: May 24, 2024
(86) PCT No.: PCT/US2024/031135

§ 371 Date: Nov. 21, 2025

Related U.S. Application Data
(60) Provisional application No. 63/505,663, filed on Jun. 1, 2023.

(51) Int. Cl. C07K 16/44; C12N 15/63; G01N 33/53; G01N 33/543; G01N 33/58

(52) U.S. Cl. CPC C07K 16/44; C12N 15/63; G01N 33/5308; G01N 33/54306; G01N 33/582; C07K 2317/24; C07K 2317/565; G01N 2333/4703

(57) ABSTRACT

The present disclosure provides antibodies that specifically bind to cyclic STAT-3 decoy oligonucleotide (CS3D). The antibodies and derivatives thereof that specifically bind to CS3D can be used in methods, such as, methods for determining pharmacokinetics of CS3D in a subject, e.g., a human receiving treatment with CS3D.

### CROSS-REFERENCE TO RELATED APPLICATIONS

This application claims the benefit of priority to U.S. Provisional Application Ser. No. 63/505,663, filed Jun. 1, 2023, the contents which are hereby incorporated by reference in their entirety.

### STATEMENT AS TO FEDERALLY SPONSORED RESEARCH

This invention was made with government support under P50 CA097190 and P41 CA196276 awarded by the National Institutes of Health. The government has certain rights in the invention.

### INCORPORATION BY REFERENCE OF SEQUENCE LISTING PROVIDED AS A SEQUENCE LISTING XML FILE

Sequence Listing is provided herewith as a Sequence Listing XML file, “UCSF-688WO_SEQ_LIST.xml”, created on Mar. 13, 2024, and having a size of 20,560 bytes. The contents of the Sequence Listing XML file are incorporated by reference herein in their entirety.

### INTRODUCTION

Signal transducer and activator of transcription proteins (STATs) are transcription factors that regulate various cellular processes such as cell proliferation, and differentiation. STAT3 has been reported to be a key factor in oncogenic signaling in the IL-6/JAK/STAT3 pathway and its hyperactivation of has been shown to occur in >70% of human cancers. However, its clinical prognosis is poor. In efforts to target STAT3, a double-stranded oligonucleotide decoy of STAT3 was developed by the Grandis group at UCSF. The cyclic STAT3 decoy (CS3D) is a double stranded DNA (dsDNA) that competitively inhibits binding of the transcription factor to its corresponding regulatory elements in genomic DNA, resulting in the inhibition of expression of target genes. Study of CS3D pharmacokinetics is important in clinical use of this molecule.

To date, only a few recombinant Abs have been developed against nucleic acid targets, with most of them targeting unique structural motifs (Ye, J.-D. et al., Proc. Natl. Acad. Sci. U.S.A 105, 82-87 (2008); Fellouse, F. A. et al., J. Mol. Biol. 373, 924-940 (2007)). However, an antibody has not been developed against CS3D.

### SUMMARY

The present disclosure provides antibodies that specifically bind to cyclic STAT3 decoy (CS3D). The antibodies that specifically bind to CS3D can be used in methods to detect and optionally quantify CS3D present in biological samples, e.g., serum or blood sample from a subject who has been administered CS3D.

The present disclosure provides an antibody that specifically binds to CS3D and competes for binding to the CS3D with (i) a first antibody comprising: a variable heavy (VH) chain comprising heavy chain complementarity determining regions 1-3 (HCDRs 1-3) of a VH chain comprising the sequence:

(SEQ ID NO: 1)
QVQLQESGGGLVQPGGSLRLSCAASGFTFSSYAMSWVRQAPGKGL
EWVSAISGSGGSTYYADSVKGRFTISRDNSKNTLYLQMNSLRAED
TAVYYCAKGLRYSSGWGQGTMVTVSS;

and a variable light (VL) chain comprising light chain complementarity determining regions 1-3 (LCDRs 1-3) of a VL chain comprising the sequence:

(SEQ ID NO: 2)
QPVLTQPPSVSKGLRQTATLTCSGNSNNVGNAGAVWLQQHQGHPP
KLLSYRNNNRPSGISERISASRSGNTASLTISGLQPEDEADYYCS
AWDSSLRVQVFGGGTKLTVL;

or (ii) a second antibody comprising: a VH chain comprising HCDRs 1-3 of a VH chain comprising the sequence:

(SEQ ID NO: 3)
QVTLQQSGPGLVKPSQTLSLTCAISGDSVSSNSAAWNWIRQSPSR
GLEWLGRTYYRSKWFTYYAASVKGRISINRDTSKNQFSLQLNSVT
PDDTAVYFCARGSGGYIDHWGQGTLVTVSS;

and a VL comprising LCDRs 1-3 of a VL chain comprising the sequence:

(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

A first antibody for CS3D is provided herein. The first antibody comprises: a variable heavy (VH) chain comprising heavy chain complementarity determining regions 1-3 (HCDRs 1-3) of a VH chain comprising the sequence:

(SEQ ID NO: 1)
QVQLQESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKGL
RYSSGWGQGTMVTVSS;

and a variable light (VL) chain comprising light chain complementarity determining regions 1-3 (LCDRs 1-3) of a VL chain comprising the sequence:

(SEQ ID NO: 2)
QPVLTQPPSVSKGLRQTATLTCSGNSNNVGNAGAVWLQQHQGHPPKLLSY
RNNNRPSGISERISASRSGNTASLTISGLQPEDEADYYCSAWDSSLRVQV
FGGGTKLTVL.

A second antibody for CS3D is provided herein. The second antibody comprises: a variable heavy (VH) chain comprising heavy chain complementarity determining regions 1-3 (HCDRs 1-3) of a VH chain comprising the sequence:

(SEQ ID NO: 3)
QVTLQQSGPGLVKPSQTLSLTCAISGDSVSSNSAAWNWIRQSPSRGLEWL
GRTYYRSKWFTYYAASVKGRISINRDTSKNQFSLQLNSVTPDDTAVYFCA
RGSGGYIDHWGQGTLVTVSS;

and a variable light (VL) chain comprising light chain complementarity determining regions 1-3 (LCDRs 1-3) of a VL chain comprising the sequence:

(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

In certain embodiments, the antibody that specifically binds to CS3D comprises: a VH chain comprising a HCDR1 comprising the amino acid sequence GFTFSSYAMS (SEQ ID NO:5), a HCDR2 comprising the amino acid sequence SAISGSGGSTY (SEQ ID NO:6), and a HCDR3 comprising the amino acid sequence GLRYSSG (SEQ ID NO:7), wherein the VH chain comprises an amino acid sequence at least 85% identical, at least 90% identical, at least 95% identical, at least 96% identical, at least 97% identical, or at least 98% identical to the amino acid sequence of SEQ ID NO:1. In certain embodiments, the antibody that further comprises: a VL chain comprising a LCDR1 comprising the amino acid sequence SGNSNNVGNAGAV (SEQ ID NO:8), a LCDR2 comprising the amino acid sequence NNNRPS (SEQ ID NO:9), and a LCDR3 comprising the amino acid sequence SAWDSSLRVQV (SEQ ID NO:10), wherein the VL chain comprises an amino acid sequence at least 85% identical, at least 90% identical, at least 95% identical, at least 96% identical, at least 97% identical, or at least 98% identical to the amino acid sequence of SEQ ID NO:2.

In certain embodiments, the antibody that specifically binds to CS3D comprises: a VH chain comprising a HCDR1 comprising the amino acid sequence GDSVSSNSAAWN (SEQ ID NO:11), a HCDR2 comprising the amino acid sequence GRTYYRSKWFTY (SEQ ID NO: 12), and a HCDR3 comprising the amino acid sequence GSGGYIDH (SEQ ID NO:13), wherein the VH chain comprises an amino acid sequence at least 85% identical, at least 90% identical, at least 95% identical, at least 96% identical, at least 97% identical, or at least 98% identical to the amino acid sequence of SEQ ID NO:3. In certain embodiments, the antibody further comprises: a VL chain comprising a LCDR1 comprising the amino acid sequence TLRSGINVDSYRIY (SEQ ID NO:14), a LCDR2 comprising the amino acid sequence DKQQGS (SEQ ID NO:15), and a LCDR3 comprising the amino acid sequence MIWHSSAVV (SEQ ID NO:16), wherein the VL chain comprises an amino acid sequence at least 85% identical, at least 90% identical, at least 95% identical, at least 96% identical, at least 97% identical, or at least 98% identical to the amino acid sequence of SEQ ID NO:4.

The antibody may be a single chain Fv (scFv), a Fab, a (Fab′) 2, an (ScFv) 2, and the like. The antibody may be an IgG. The antibody may also be in a pharmaceutically acceptable excipient.

The antibody may be labeled. The antibody may be attached to a solid support.

Nucleic acids provided herein encode one or more antibodies that are described herein. Cells comprising such nucleic acids are also provided herein. Kits comprising one or more described herein are disclosed. The kits optionally also include instructional materials teaching the use of the antibody for measuring pharmacokinetics of CS3D.

### BRIEF DESCRIPTION OF THE DRAWINGS

FIG. 1. Schematic of RAPID biopanning for the identification of rare high-affinity binders against challenging targets. RAPID biopanning follows a Label-Profile-Sort-Screen pipeline. First, phage libraries from rounds of biopanning are individually FITC (yellow star) labeled and each incubated with Ag-Beads (beige antigen, grey bead). Subsequent analysis with flow cytometry profiles the progression of the biopanning campaign and accurately identifies the most Ag-Ab enriched round. For ph-Fab libraries that exhibit extremely low frequencies of high-affinity Abs against Ags, fluorescent activated sorting is performed to isolate specific populations of ph-Fabs that contain higher frequencies of high affinity binders. A novel BLI method is subsequently employed to rapidly screen candidate clones in a discriminatory matter, thereby prioritizing promising hits for further investigations. For comparison, standard biopanning using magnetic beads (beige antigen, red bead) is depicted in the upper left corner.

FIGS. 2A-2F. Flow cytometry of four ph-Fabs with ARS1620-V7 (VVVGACGVGK (SEQ ID NO:17)). (A) Pull down titers of ph-P1A4, ph-P1C1, ph-P1H6, and ph-P2F11. FITC labeled and unlabeled ph-Fab exhibit similar phage titers. (B) FITC labeling of M13, ph-P1A4, and ph-P2F11. (C) Flow cytometry analysis of ph-P1C1, ph-P1H6, ph-P2F11, and ph-P1A4 bound to Ag-bead. (D) Normalized median fluorescent intensity (MFI) of flow cytometry data from (D). Importantly, the normalized MFI trends match with the labeled titer trends in (A), ensuring ph-Fabs are labeled with no biases and interactions between Ag and displayed Fabs are not significantly affected by FITC labeling. (E) RAPID flow cytometry profiling with different percentages of ph-P1A4 and M13 (FITC labeled). (F) An increase in ph-P1A4 concentration shows an increase in normalized MFI with an observable shift from flow cytometry starting at 10 percent ph-P1A4.

FIG. 3 depicts RAPID fluorescent activated sorting of fluorescently labeled ph-Fab libraries. RAPID fluorescent activated sorting allows for the isolation of higher-affinity ph-Fab populations. Iterative rounds of sorting can result in the enrichment of ph-Fab libraries to be re-biased towards Ag-Fab binding where severe growth/display propensity biases occur in standard biopanning. ph-Fab libraries are first FITC labeled and incubated with Ag-Beads. Non-binding phage are removed by washing, and Ag-Bead-fluorescent ph-Fab complex is sorted and bead populations containing high fluorescence are isolated.

FIGS. 4A-4D. Biolayer Interferometry Antibody Screen (BIAS). (A) BIAS scheme. Representative BLI sensograms of each possible scenario (Hit, False positive, Negative) are shown on the right. (i) Association-1: Biological tips bound with Ag are transferred to crude PPEs induced for expression, (ii) Association-2: tips are transferred to wells containing identical PPE sample from Association-1+anti-myc IgG, (iii) Dissociation: tips (ii) are transferred to wells with no PPE and no IgG. The Association-2 step distinguishes hits versus false positives. (B)-(D) BIAS with P1A4 spiked samples. (B) P1A4 (250 nM) spiked in PBS. A signature linear curve is observed in Assco-2 where 9E10 is present, while this is not observed with the control containing no 9E10. (C) P1A4 (250 nM) spiked in TG1-PPE. Results are similar as (C). (D) Concentration series of P1A4 spiked in TG1-PPE (10⁻³⁰⁰ nM).

FIGS. 5A to 5E antibody discovery campaign of Carboxyl terminus of Hsp-70 interacting protein (CHIP). (A) PhAB flow from Rounds 1-4. Round 3 shows the highest fluorescent distribution shift. (B) Normalized MFI of PhAB flow of rounds 1-4. Round 3 shows a normalized MFI of 2.11 which suggest a hit between 10 and 50 percent. (C) Comparison of BIAS k_{off} and BLI measured k_{off} (at 500 nM) of BIAS hits (*=9.40×10⁻⁶). Despite F1 and D11 not showing fully saturated curves BIAS k_{off} and BLI measured k_{off} show reasonable correlation in rankings and values. (D) Round 3 clones screened with dot blot. Hits are circled in blue with BIAS hits circled with a thicker boarder. (E) Round 3 clones screened with BIAS. Hits, False positives, and Negatives are shown according to the legend.

FIGS. 6A to 6F antibody discovery campaign of Carboxyl terminus of Hsp-70 interacting protein (CHIP). (A)-(F) Full BIAS curves for six candidate clones. Clones with potentially inaccurate k_{off} predictions are flagged (*-low expression #-unsatruated association-1,!-poor exponential fit for dissociation).

FIGS. 7A to 7F antibody discovery campaign of CS3D. (A) PhAB flow of CS3D biopanning campaign, rounds 1-5. No significant increase in fluorescent distribution is observed. (B) Zoom in overlay of PhAB flow of rounds 1-5. A shoulder distribution is observed. (C) Normalized MFI of PhAB flow of rounds 1-5. (D) Normalized MFI and population percentage of shoulder population gated from round 1-5. Both normalized MFI and population percentage are highest at round 4 indicating the highest probability of promising high-affinity ph-Fab are included in round 4. (E) Shoulder population gate from round 4 PhAB sorting (orange), against Ag-Bcad control (magenta). Roughly 4% of the total population of round 4 includes the shoulder population with potential rare high-affinity ph-Fab. (F) BIAS k_{off}s of sorted (top 4%) and unsorted (bottom 96%) randomly chosen clones. The sorted population contains ~2-fold lower BIAS k_{off}s suggesting more promising binders exist in the sorted versus unsorted population of ph-Fab.

FIG. 8 biotin-FITC bound to streptavidin beads in increasing concentrations. A maximal distribution shift is observed starting at 1 μM biotin-FITC immobilization with no overlap to the negative control with no phage.

FIGS. 9A to 9D depict RAPID Flow cytometry enrichment profiling with fluorescently labeled ph-Fab libraries. (A) Depending on the growth/display propensities of higher affinity ph-Fabs, iterative rounds of biopanning result in different relative populations of ph-Fab binders. (B) Where growth/display levels are similar between ph-Fab library members, flow distributions show an increase in normalized MFI while the coefficient variability is similar (CV). (C) Where high-affinity ph-Fabs exhibit inferior growth/display propensities, MFI increase is halted prematurely, and CV increases as weaker binders are more prevalent. (D) Where high-affinity ph-Fabs also exhibit superior growth/display propensities, an increase in MFI and a decrease CV is observed.

FIG. 10 depicts a BATCH categorization, ranking, and output scheme.

FIG. 11 depicts a BATCH algorithm scheme.

FIGS. 12A to 12C optimization of phage labeling with NHS-FITC and M13 phage. (A) Fluorescent signal is increased as FITC labelling concentration is increased FITC labeling. (B) Washing optimizations show three washing steps are sufficient for complete depletion of non-reacted NHS-FITC. (C) FITC labeling is saturated after 1 hour reaction time.

FIG. 13 simple guide to ranking flagged clones from BIAS screens. For Fab candidates that do not fully saturate or show very low expression levels (very low Δnm in Assoc-1), Assoc-2 curves are better indications of binding affinity than BIAS k_{off}. Roughly, lower true Assoc-2 slopes indicate higher affinity.

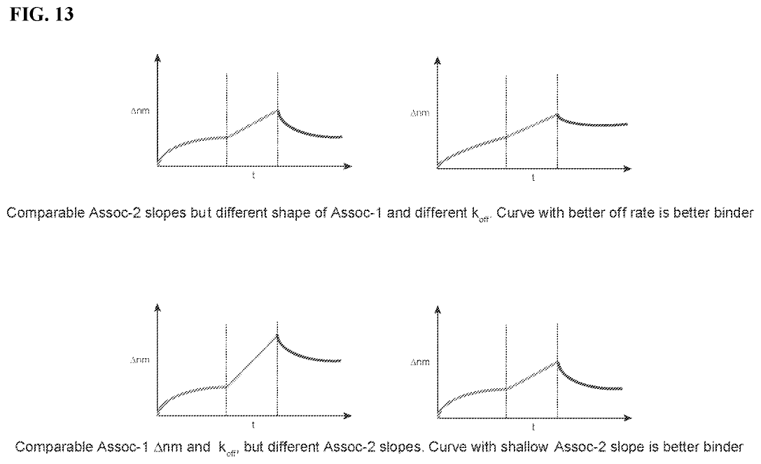

### Definitions

“Derived from” in the context of an amino acid sequence or polynucleotide sequence is meant to indicate that the polypeptide or nucleic acid has a sequence that is based on that of a reference polypeptide or nucleic acid, and is not meant to be limiting as to the source or method in which the protein or nucleic acid is made.

An “anti-CS3D antibody” refers to an antibody that binds CS3D. An anti-CS3D antibody may bind to CS3D with a K_{D} less than about 10⁻⁷M, less than about 10⁻⁸, less than about 10⁻⁹, less than about 10⁻¹⁰, less than about 10⁻¹¹, or less than about 10⁻¹² or less. In certain embodiments, “high affinity” antibodies have a K_{D} of 100 nM or less.

The terms “polypeptide”, “peptide”, and “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids, which include, but are not limited to those listed in 37 CFR (§ 1.822 (b) (4)). Furthermore, it should be noted that a dash at the beginning or end of an amino acid residue sequence indicates either a peptide bond to a further sequence of one or more amino acid residues or a covalent bond to a carboxyl or hydroxyl end group. However, the absence of a dash should not be taken to mean that such peptide bonds or covalent bond to a carboxyl or hydroxyl end group is not present, as it is conventional in representation of amino acid sequences to omit such.

The term “antibody” (also used interchangeably with “immunoglobulin”) encompasses polyclonal and monoclonal antibody preparations where the antibody may be of any class of interest (e.g., IgM, IgG, and subclasses thereof), as well as preparations including hybrid antibodies, altered antibodies, F(ab′) 2 fragments, F(ab) molecules, Fv fragments, scFv fragments, single chain antibodies, single domain antibodies, chimeric antibodies, humanized antibodies, and functional fragments thereof which exhibit binding properties of the parent antibody molecule. The antibodies may be conjugated to other moieties, such as, labels, and/or may be bound to a support (e.g., a solid support), such as a polystyrene plate or bead, test strip, and the like.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region is composed of a “framework” region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, (1991) and Lefranc et al. IMGT, the international ImMunoGeneTics information system®. Nucl. Acids Res., (2005) 33: D593-D597)). A detailed discussion of the IMGTS system, including how the IMGTS system was formulated and how it compares to other systems, is provided on the World Wide Web at imgt.cines.fr/textes/IMGTScientificChart/Numbering/IMGTnumberingsTable.html. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat et al, supra, unless otherwise indicated. The three light chain CDRs, as used herein, are also referred to as “LCDR1”, “LCDR2”, and “LCDR3”. The three heavy chain variable CDRs, as used herein, are also referred to as “HCDR1”, “HCDR2”, and “HCDR3”.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies encompass intact immunoglobulins as well as a number of well characterized fragments produced by digestion with various peptidases. Thus, for example, pepsin digests an antibody below the disulfide linkages in the hinge region to produce F(ab)′₂, a dimer of Fab which itself is a light chain joined to VH—CH1 by a disulfide bond. The F(ab)′₂ may be reduced under mild conditions to break the disulfide linkage in the hinge region thereby converting the (Fab′) 2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies, including, but are not limited to, Fab′₂, IgG, IgM, IgA, scFv, dAb, nanobodies, unibodies, and diabodies.

Antibodies and fragments of the present disclosure encompass those that are bispecific. Bispecific antibodies or fragments can be of several configurations. For example, bispecific antibodies may resemble single antibodies (or antibody fragments) but have two different antigen binding sites (variable regions). Bispecific antibodies may be produced by chemical techniques (Kranz et al. (1981) Proc. Natl. Acad. Sci., USA, 78:5807), by “polydoma” techniques (see, e.g., U.S. Pat. No. 4,474,893), or by recombinant DNA techniques. Bispecific antibodies may have binding specificities for at least two different epitopes, at least one of which is an epitope of CS3D.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, MD (1987).

As used herein, the terms “immunological binding” and “immunological binding properties” refer to the non-covalent interactions of the type which occur between an immunoglobulin molecule and an antigen for which the immunoglobulin is specific. The strength or affinity of immunological binding interactions can be expressed in terms of the dissociation constant (K_{D}) of the interaction, wherein a smaller K_{D} represents a greater affinity. Immunological binding properties of selected polypeptides can be quantified using methods well known in the art. One such method entails measuring the rates of antigen binding site/antigen complex formation and dissociation, wherein those rates depend on the concentrations of the complex partners, the affinity of the interaction, and on geometric parameters that equally influence the rate in both directions. Thus, both the “on rate constant” (kₒₙ) and the “off rate constant” (k_{off}) can be determined by calculation of the concentrations and the actual rates of association and dissociation. The ratio of k_{off}/kₒₙ enables cancellation of all parameters not related to affinity and is thus equal to the equilibrium dissociation constant K_{D} (see, generally, Davies el al. Ann. Rev. Biochem. 1990, 59:439-15 473).

An “epitope” is a site on an antigen (e.g., CS3D) to which an antibody binds.

“Isolated” refers to an entity of interest that is in an environment different from that in which the compound may naturally occur or initially produced in. An “isolated” compound (e.g., an “isolated” antibody) is separated from all or some of the components that accompany it and may be substantially enriched, e.g., may be purified so that the compound is at least about 70% pure, at least about 80% pure, at least about 90% pure, at least about 95% pure, at least about 98% pure, at least about 99%, or greater than 99% pure, or free of impurities, contaminants, and/or components other than the compound. “Isolated” also refers to the state of a compound separated from all or some of the components that accompany it during manufacture (e.g., chemical synthesis, recombinant expression, culture medium, and the like).

A single chain Fv (“scFv”) polypeptide is a covalently linked V_{H}::V_{L} heterodimer which may be expressed from a nucleic acid including Vu- and VL-encoding sequences either joined directly or joined by a peptide-encoding linker (Huston, et al. (1988) Proc. Nat. Acad. Sci. USA, 85:5879-5883). A number of structures are available for converting the light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

Recombinant design methods may be used to develop suitable chemical structures (linkers) for converting two heavy and light polypeptide chains from an antibody variable region into a scFv molecule which will fold into a three-dimensional structure that is substantially similar to native antibody structure.

Design criteria include determination of the appropriate length to span the distance between the C-terminal of one chain and the N-terminal of the other, wherein the linker is generally formed from small hydrophilic amino acid residues that do not tend to coil or form secondary structures. Such methods have been described in the art. See, e.g., U.S. Pat. Nos. 5,091,513 and 5,132,405 to Huston et al.; and U.S. Pat. No. 4,946,778 to Ladner et al.

In this regard, the first general step of linker design involves identification of plausible sites to be linked. Appropriate linkage sites on each of the VH and VL polypeptide domains include those which will result in the minimum loss of residues from the polypeptide domains, and which will necessitate a linker comprising a minimum number of residues consistent with the need for molecule stability. A pair of sites defines a “gap” to be linked. Linkers connecting the C-terminus of one domain to the N-terminus of the next generally comprise hydrophilic amino acids which assume an unstructured configuration in physiological solutions and may be free of residues having large side groups which might interfere with proper folding of the VH and VL chains. Thus, suitable linkers generally comprise polypeptide chains of alternating sets of glycine and serine residues, and may include glutamic acid and lysine residues inserted to enhance solubility. One particular linker has the amino acid sequence (Gly₄Ser)₃ (SEQ ID NO:17). Another example of a suitable linker is a linker that has the amino acid sequence comprising 2 or 3 repeats of [(Ser)₄Gly] (SEQ ID NO:20), such as [(Ser)₄Gly] 3 (SEQ ID NO: 21), and the like. Nucleotide sequences encoding such linker moieties can be readily provided using various oligonucleotide synthesis techniques known in the art (see, e.g., Sambrook, supra.).

The phrase “specifically binds to” or “specifically immunoreactive with”, when referring to an antibody that specifically binds to an antigen refers to a binding reaction which is determinative of the presence of the antigen in the presence of a heterogeneous population of other molecules, proteins, DNA, and other biologics. Thus, under designated immunoassay conditions, the specified antibodies bind to a particular antigen and do not bind in a significant amount to other molecules present in the sample. Specific binding to an antigen under such conditions may require an antibody that is selected for its specificity for the antigen. A variety of immunoassay formats may be used to select antibodies specifically immunoreactive with a particular antigen. For example, solid-phase enzyme-linked immunosorbent assay (ELISA) immunoassays are routinely used to select monoclonal antibodies specifically immunoreactive with an antigen. See Harlow and Lane (1988) Antibodies, A Laboratory Manual, Cold Spring Harbor Publications, New York, for a description of immunoassay formats and conditions that can be used to determine specific immunoreactivity.

The term “conservative substitution” is used in reference to proteins or peptides to reflect amino acid substitutions that do not substantially alter the activity (specificity or binding affinity) of the molecule. Typically, conservative amino acid substitutions involve substituting one amino acid for another amino acid with similar chemical properties (e.g., charge or hydrophobicity). The following six groups each contain amino acids that are typical conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### DETAILED DESCRIPTION

The present disclosure provides antibodies that specifically bind to CS3D. CS3D is a cyclic STAT3 double stranded, oligonucleotide decoy that has the following structure (SEQ ID NO: 19):

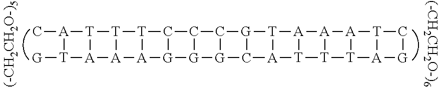

CS3D binds toSTAT3 and inhibits expression of downstream STAT3 induced genes.

### I. CS3D-Binding Antibodies

In certain embodiments, the anti-CS3D antibodies disclosed herein bind to CS3D with a K_{D} of about 10 nM to about 100 nM.

The present disclosure provides an antibody that specifically binds to CS3D and competes for binding to the CS3D with (i) a first antibody comprising: a variable heavy (VH) chain comprising heavy chain complementarity determining regions 1-3 (HCDRs 1-3) of a VH chain comprising the sequence:

(SEQ ID NO: 1)
QVQLQESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKGL
RYSSGWGQGTMVTVSS;

and a variable light (VL) chain comprising light chain complementarity determining regions 1-3 (LCDRs 1-3) of a VL chain comprising the sequence:

(SEQ ID NO: 2)
QPVLTQPPSVSKGLRQTATLTCSGNSNNVGNAGAVWLQQHQGHPPKLLSY
RNNNRPSGISERISASRSGNTASLTISGLQPEDEADYYCSAWDSSLRVQV
FGGGTKLTVL;

or

(ii) a second antibody comprising: a VH chain comprising HCDRs 1-3 of a VH chain comprising the sequence:

(SEQ ID NO: 3)
QVTLQQSGPGLVKPSQTLSLTCAISGDSVSSNSAAWNWIRQSPSRGLEWL
GRTYYRSKWFTYYAASVKGRISINRDTSKNQFSLQLNSVTPDDTAVYFCA
RGSGGYIDHWGQGTLVTVSS;

and a VL comprising LCDRs 1-3 of a VL chain comprising the sequence:

(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

Any suitable approach for determining whether a first antibody competes with a second antibody for binding to CS3D may be employed. Whether a first antibody “competes with” a second antibody for binding to a compound may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labelled. If the labeled antibody and the unlabeled antibody bind to separate and discrete sites on the compound, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the compound by about 30% or more, about 40% or more, about 50% or more, about 60% or more, about 70% or more, about 80% or more, about 85% or more, about 90% or more, about 95% or more, or about 99% or more. Details of procedures for carrying out such competition assays are well known in the art and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the target epitope may be titrated. The results may be plotted, and the concentrations necessary to achieve the desired degree of binding inhibition may be compared.

A first antibody for CS3D is provided herein. The first antibody comprises: a variable heavy (VH) chain comprising heavy chain complementarity determining regions 1-3 (HCDRs 1-3) of a VH chain comprising the sequence:

(SEQ ID NO: 1)
QVQLQESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKGL
RYSSGWGQGTMVTVSS;

and a variable light (VL) chain comprising light chain complementarity determining regions 1-3 (LCDRs 1-3) of a VL chain comprising the sequence:

(SEQ ID NO: 2)
QPVLTQPPSVSKGLRQTATLTCSGNSNNVGNAGAVWLQQHQGHPPKLLSY
RNNNRPSGISERISASRSGNTASLTISGLQPEDEADYYCSAWDSSLRVQV
FGGGTKLTVL.

In certain embodiments, the HCDR1 comprises the amino acid sequence GFTFSSYAMS (SEQ ID NO:5), the HCDR2 comprises the amino acid sequence SAISGSGGSTY (SEQ ID NO:6), and the HCDR3 comprises the amino acid sequence GLRYSSG (SEQ ID NO:7) and the LCDR1 comprises the amino acid sequence SGNSNNVGNAGAV (SEQ ID NO:8), the LCDR2 comprises the amino acid sequence NNNRPS (SEQ ID NO: 9), and the LCDR3 comprises the amino acid sequence SAWDSSLRVQV (SEQ ID NO:10).

A second antibody for CS3D is provided herein. The second antibody comprises: a variable heavy (VH) chain comprising heavy chain complementarity determining regions 1-3 (HCDRs 1-3) of a VH chain comprising the sequence:

(SEQ ID NO: 3)
QVTLQQSGPGLVKPSQTLSLTCAISGDSVSSNSAAWNWIRQSPSRGLEWL
GRTYYRSKWFTYYAASVKGRISINRDTSKNQFSLQLNSVTPDDTAVYFCA
RGSGGYIDHWGQGTLVTVSS;

and a variable light (VL) chain comprising light chain complementarity determining regions 1-3 (LCDRs 1-3) of a VL chain comprising the sequence:

(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

In certain embodiments, the HCDR1 comprises the amino acid sequence GDSVSSNSAAWN (SEQ ID NO: 11), the HCDR2 comprises the amino acid sequence GRTYYRSKWFTY (SEQ ID NO:12), and the HCDR3 comprises the amino acid sequence GSGGYIDH (SEQ ID NO:13) and the LCDR1 comprises the amino acid sequence TLRSGINVDSYRIY (SEQ ID NO:14), the LCDR2 comprises the amino acid sequence DKQQGS (SEQ ID NO:15), and the LCDR3 comprises the amino acid sequence MIWHSSAVV (SEQ ID NO:16).

In certain embodiments, the antibody that specifically binds to CS3D comprises: a VH chain comprising a HCDR1 comprising the amino acid sequence GFTFSSYAMS (SEQ ID NO:5), a HCDR2 comprising the amino acid sequence SAISGSGGSTY (SEQ ID NO:6), and a HCDR3 comprising the amino acid sequence GLRYSSG (SEQ ID NO:7), wherein the VH chain comprises an amino acid sequence at least 85% identical, at least 90% identical, at least 95% identical, at least 96% identical, at least 97% identical, or at least 98% identical to the amino acid sequence of SEQ ID NO:1. In certain embodiments, the antibody that further comprises: a VL chain comprising a LCDR1 comprising the amino acid sequence SGNSNNVGNAGAV (SEQ ID NO:8), a LCDR2 comprising the amino acid sequence NNNRPS (SEQ ID NO:9), and a LCDR3 comprising the amino acid sequence SAWDSSLRVQV (SEQ ID NO:10), wherein the VL chain comprises an amino acid sequence at least 85% identical, at least 90% identical, at least 95% identical, at least 96% identical, at least 97% identical, or at least 98% identical to the amino acid sequence of SEQ ID NO:2.

In certain embodiments, the antibody that specifically binds to CS3D comprises: a VH chain comprising a HCDR1 comprising the amino acid sequence GDSVSSNSAAWN (SEQ ID NO:11), a HCDR2 comprising the amino acid sequence GRTYYRSKWFTY (SEQ ID NO:12), and a HCDR3 comprising the amino acid sequence GSGGYIDH (SEQ ID NO:13), wherein the VH chain comprises an amino acid sequence at least 85% identical, at least 90% identical, at least 95% identical, at least 96% identical, at least 97% identical, or at least 98% identical to the amino acid sequence of SEQ ID NO:3. In certain embodiments, the antibody further comprises: a VL chain comprising a LCDR1 comprising the amino acid sequence TLRSGINVDSYRIY (SEQ ID NO:14), a LCDR2 comprising the amino acid sequence DKQQGS (SEQ ID NO:15), and a LCDR3 comprising the amino acid sequence MIWHSSAVV (SEQ ID NO:16), wherein the VL chain comprises an amino acid sequence at least 85% identical, at least 90% identical, at least 95% identical, at least 96% identical, at least 97% identical, or at least 98% identical to the amino acid sequence of SEQ ID NO:4.

In certain embodiments, the amino acid sequences of various anti-CS3D antibodies, as well as each CDR and framework region, are shown below:

| Region | 4E4 Sequence |
|---|---|
| VL | QPVLTQPPSVSKGLRQTATLTCSGNSNNVGNAGAVWLQQHQGHPPKLLSYRNNNRPS |
| | GISERISASRSGNTASLTISGLQPEDEADYYCSAWDSSLRVQVFGGGTKLTVL |
| | (SEQ ID NO: 2) |
| VH | QVQLQESGGGLVQPGGSLRLSCAASGFTFSSYAMSWVRQAPGKGLEWVSAISGSGGS |
| | TYYADSVKGRFTISRDNSKNTLYLQMNSLRAEDTAVYYCAKGLRYSSGWGQGTMV |
| | TVSS (SEQ ID NO: 1) |
| LCDR1 | SGNSNNVGNAGAV (SEQ ID NO: 8) |
| LCDR2 | NNNRPS (SEQ ID NO: 9) |
| LCDR3 | SAWDSSLRVQV (SEQ ID NO: 10) |
| HCDR1 | GFTFSSYAMS (SEQ ID NO: 5) |
| HCDR2 | SAISGSGGSTY (SEQ ID NO: 6) |
| HCDR3 | GLRYSSG (SEQ ID NO: 7) |
| Region | SP1-B3 Sequence |
| VL | QAVLTQPSSLSASPGASASLTCTLRSGINVDSYRIYWYQQKPGSPPQYLLRYKSDSDK |
| | QQGSGVPSRFSGSKDASANAGILLISGLQSEDEADYYCMIWHSSAVVFGGGTKLTVL |
| | (SEQ ID NO: 4) |
| VH | QVTLQQSGPGLVKPSQTLSLTCAISGDSVSSNSAAWNWIRQSPSRGLEWLGRTYYRSK |
| | WFTYYAASVKGRISINRDTSKNQFSLQLNSVTPDDTAVYFCARGSGGYIDHWGQGT |
| | LVTVSS (SEQ ID NO: 3) |
| LCDR1 | TLRSGINVDSYRIY (SEQ ID NO: 14) |
| LCDR2 | DKQQGS (SEQ ID NO: 15) |
| LCDR3 | MIWHSSAVV (SEQ ID NO: 16) |
| HCDR1 | GDSVSSNSAAWN (SEQ ID NO: 11) |
| HCDR2 | GRTYYRSKWFTY (SEQ ID NO: 12) |
| HCDR3 | GSGGYIDH (SEQ ID NO: 13) |

The framework regions in the sequences of VH and VL chains are underlined and the CDRs are italisized.

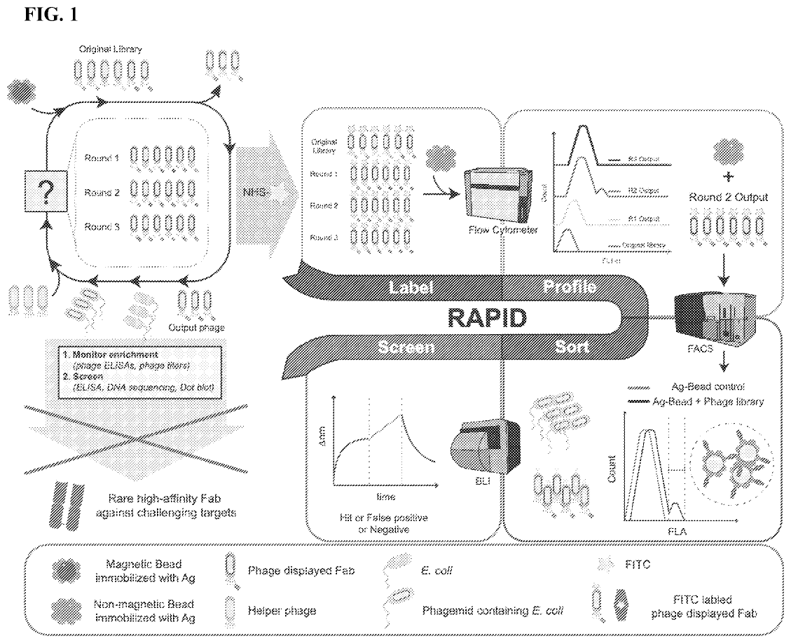

It will be appreciated that the amino acid sequence of a CDR can also be defined using alternative systems, which will be readily apparent to and applied by the ordinarily skilled artisan (see, “Sequences of Proteins of Immunological Interest,” E. Kabat et al., U.S. Department of Health and Human Services, (1991); and Lefranc et al. IMGT, the international ImMunoGeneTics information system. Nucl. Acids Res., (2005) 33: D593-D597)). A detailed discussion of the IMGTS system, including how the IMGTS system was formulated and how it compares to other systems, is provided on the World Wide Web at imgt.cines.fr/textes/IMGTScientificChart/Numbering/IMGTnumberingsTable.html. As seen in FIG. 1, CDRs are demarcated for each antibody in their respective columns and labels. All amino acid sequences of CDR in the present disclosure are defined according to Kabat et al., supra, unless otherwise indicated.

Using the teachings and the sequence information provided herein, the variable light and variable heavy chains can be joined directly or through a linker (e.g., (Gly₄Ser)₃, SEQ ID NO:17) to form a single-chain Fv antibody. The various CDRs and/or framework regions can be used to form human antibodies, chimeric antibodies, antibody fragments, polyvalent antibodies, and the like.

Anti-CS3D antibodies of the present disclosure have a binding affinity (K_{D}) for a CS3D protein of at least 10⁻⁷M, at least 10⁻⁸ M, at least 10⁻⁹ M, at least 10⁻¹⁰ M, at least 10⁻¹¹ M, or at least 10⁻¹² M. Some examples of K_{D}s (M⁻¹) for CS3D fall in the following ranges: between about 2×10⁻¹² to about 5× 10⁻¹⁰, between about 5×10⁻¹⁰ to about 1×10⁻⁹, between 1×10⁻⁹ to 5×10⁻⁹, between 5×10⁹ to 1× 10 8, between 4×10⁻⁹ to 2×10⁻⁸.

In some cases, the antibody has a K_{D} with a CS3D of about 5 nM or less.

The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

In certain embodiments, the antibody further comprises a heavy chain constant region and a light chain constant region of a human IgG antibody, e.g., human IgG1 antibody. In certain embodiments, the antibody molecules disclosed herein include a heavy chain comprising a variable heavy chain region as provided herein and a human IgG1 constant region having the amino acid sequence sequence set forth in UniProt: P01857-1, version 1. In certain embodiments, the antibody molecules disclosed herein include a light chain comprising a variable light chain region as provided herein and a human light chain constant region. In certain embodiments, the human light chain constant region is a human kappa light chain constant region having the amino acid set forth in UniProtKB/Swiss-Prot: P01834.2. I.

### III. Preparation of Anti-CS3D Antibodies

### Recombinant Expression of Anti-CS3D Antibodies

Using the information provided herein, the antibodies of the present disclosure are prepared using standard techniques well known to those of skill in the art.

For example, the polypeptide sequences provided herein can be used to determine appropriate nucleic acid sequences encoding the anti-CS3D antibodies and the nucleic acids sequences then used to express one or more CS3D antibodies. The nucleic acid sequence(s) can be optimized to reflect particular codon “preferences” for various expression systems according to standard methods well known to those of skill in the art.

Using the sequence information provided, the nucleic acids may be synthesized according to a number of standard methods known to those of skill in the art. Oligonucleotide synthesis can be carried out on commercially available solid phase oligonucleotide synthesis machines (Needham-VanDevanter et al. (1984) Nucleic Acids Res. 12:6159-6168) or manually synthesized using, for example, the solid phase phosphoramidite triester method described by Beaucage et al. (1981) Tetrahedron Letts. 22 (20): 1859-1862.

Once a nucleic acid encoding an antibody of the present disclosure is synthesized it can be amplified and/or cloned according to standard methods. Molecular cloning techniques to achieve these ends are known in the art. A wide variety of cloning and in vitro amplification methods suitable for the construction of recombinant nucleic acids are known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, CA (Berger); Sambrook et al. (1989) Molecular Cloning-A Laboratory Manual (2nd ed.) Vol. 1-3, Cold Spring Harbor Laboratory, Cold Spring Harbor Press, NY, (Sambrook); and Current Protocols in Molecular Biology, F.M. Ausubel et al., eds., Current Protocols, a joint venture between Greene Publishing Associates, Inc. and John Wiley & Sons, Inc., (1994 Supplement) (Ausubel). Methods of producing recombinant immunoglobulins are also known in the art. See, Cabilly, U.S. Pat. No. 4,816,567; and Queen et al. (1989) Proc. Nat'l Acad. Sci. USA 86:10029-10033.

Examples of techniques sufficient to direct persons of skill through in vitro amplification methods, including the polymerase chain reaction (PCR), the ligase chain reaction (LCR), Qβ-replicase amplification and other RNA polymerase mediated techniques are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, CA (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; and Barringer et al. (1990) Gene 89, 117. Improved methods of cloning in vitro amplified nucleic acids are described in Wallace et al., U.S. Pat. No. 5,426,039.

Once the nucleic acid for an antibody of the present disclosure is isolated and cloned, one can express the gene in a variety of recombinantly engineered cells known to those of skill in the art. Examples of such cells include bacteria, yeast, filamentous fungi, insect (especially employing baculoviral vectors), plant, and mammalian cells. It is expected that those of skill in the art are knowledgeable in the numerous expression systems available for expression of antibodies.

In brief summary, the expression of natural or synthetic nucleic acids encoding antibodies of the present disclosure will typically be achieved by operably linking a nucleic acid encoding the antibody to a promoter (which is either constitutive or inducible), and incorporating the construct into an expression vector. The vectors can be suitable for replication and integration in prokaryotes, eukaryotes, or both. Typical cloning vectors contain transcription and translation terminators, initiation sequences, and promoters useful for regulation of the expression of the nucleic acid encoding the anti-CS3D antibody. The vectors optionally comprise generic expression cassettes containing at least one independent terminator sequence, sequences permitting replication of the cassette in both eukaryotes and prokaryotes, i.e., shuttle vectors, and selection markers for both prokaryotic and eukaryotic systems. See Sambrook et al (1989) supra.

To obtain high levels of expression of a cloned nucleic acid it is common to construct expression plasmids which typically contain a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky (1984) J. Bacteriol., 158:1018-1024, and the leftward promoter of phage lambda (PL) as described by Herskowitz and Hagen (1980) Ann. Rev. Genet., 14:399-445 and the L-arabinose (araBAD) operon (Better (1999) Gene Exp Systems pp95-107 Academic Press, Inc., San Diego, CA). The inclusion of selection markers in DNA vectors transformed in E. coli is also useful. Examples of such markers include genes specifying resistance to ampicillin, tetracycline, or chloramphenicol. See Sambrook et al (1989) supra for details concerning selection markers, e.g., for use in E. coli.

Expression systems for expressing antibodies of the present disclosure are available using, for example, E. coli, Bacillus sp. (see, e.g., Palva, et al. (1983) Gene 22:229-235; Mosbach et al. (1983) Nature, 302:543-545), and Salmonella. E. coli systems may also be used.

The antibodies produced by prokaryotic cells may require exposure to chaotropic agents for proper folding. During purification from, e.g., E. coli, the expressed protein is optionally denatured and then renatured. This can be accomplished, e.g., by solubilizing the bacterially produced antibodies in a chaotropic agent such as guanidine HCL. The antibody is then renatured, either by slow dialysis or by gel filtration (see, e.g., U.S. Pat. No. 4,511,503). Alternatively, nucleic acid encoding the antibodies of the present disclosure may be operably linked to a secretion signal sequence such as pelB so that the antibodies are secreted into the medium in correctly-folded form (Better et al (1988) Science 240:1041-1043).

Methods of transfecting and expressing genes in mammalian cells are known in the art (see e.g., Birch and Racher Adv. Drug Deliv. Rev. 2006, 58:671-685). Transducing cells with nucleic acids can involve, for example, incubating viral vectors containing the nucleic acids with cells within the host range of the vector (see, e.g., Goeddel (1990) Methods in Enzymology, vol. 185, Academic Press, Inc., San Diego, CA or Krieger (1990) Gene Transfer and Expression--A Laboratory Manual, Stockton Press, New York, N. Y. and the references cited therein).

The culture of cells used in the present disclosure, including cell lines and cultured cells from tissue or blood samples is well known in the art (see, e.g., Freshney (1994) Culture of Animal Cells, a Manual of Basic Technique, third edition, Wiley-Liss, N. Y. and the references cited therein).

Techniques for using and manipulating antibodies are found in Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, CA, and references cited therein; Goding (1986) Monoclonal Antibodies: Principles and Practice (2d ed.) Academic Press, New York, NY; and Kohler and Milstein (1975) Nature 256:495-497.

The nucleic acids may be subcloned into the expression vector pUC119mycHis (Tomlinson et al. (1996) J. Mol. Biol., 256:813-817) or pSYN3, resulting in the addition of a hexahistidine tag at the C-terminal end of the scFv to facilitate purification.

The present disclosure provides an isolated nucleic acid comprising a nucleotide sequence encoding an antibody of the present disclosure. The present disclosure provides an isolated nucleic acid comprising a nucleotide sequence encoding an amino acid sequence of a V_{H} of a subject antibody. The present disclosure provides an isolated nucleic acid comprising a nucleotide sequence encoding an amino acid sequence of a V_{L} of a subject antibody. The present disclosure provides an isolated nucleic acid comprising a nucleotide sequence encoding an amino acid sequence of a VH and a VL of a subject antibody. In some instances, a subject nucleic acid comprises a nucleotide sequence encoding VH CDR1, CDR2, and CDR3 of a subject antibody and/or a VL CDR1, CDR2, and CDR3 of a subject antibody.

The nucleic acid can be a recombinant vector, as described above, which provides for amplification and/or expression (synthesis) of the encoded antibody. The recombinant vector can be suitable for expression in prokaryotic and/or eukaryotic cells.

The present disclosure also provides a cell, e.g., a genetically modified cell, that comprises a subject nucleic acid. A subject genetically modified cell can be a prokaryotic cell (e.g., a bacterial cell); or a eukaryotic cell (e.g., an insect cell; a mammalian cell, such as a mammalian cell line suitable for in vitro cell culture; a yeast cell; etc.), where the cell may produce the encoded antibody.

### IV. Modification of Antibodies

Creation of (scFv′)2 Homodimers

To create (scFv′)₂ antibodies, two scFvs are joined, either through a linker (e.g., a carbon linker, a peptide, etc.) or through a disulfide bond between, for example, two cysteines. Thus, for example, to create disulfide linked scFv, a cysteine residue can be introduced by site directed mutagenesis between a myc tag and a hexahistidine tag at the carboxy-terminus of an antibody. Introduction of the correct sequence can be verified by DNA sequencing. The construct may be in pUC119, so that the pelB leader directs expressed scFv to the periplasm. Expressed scFv has the myc tag at the C-terminus, followed by two glycines, a cysteine, and then 6 histidines to facilitate purification by IMAC. After disulfide bond formation between the two cysteine residues, the two scFv can be separated from each other by 26 amino acids (two 11 amino acid myc tags and three repeats of a unit with 4 glycines plus one serine). An scFv expressed from this construct, purified by IMAC may predominantly comprise monomeric scFv. To produce (scFv′)₂ dimers, the cysteine can be reduced by incubation with 1 mM beta-mercaptoethanol, and half of the scFv blocked by the addition of DTNB. Blocked and unblocked scFvs can be incubated together to form (scFv′)₂ and the resulting material can optionally be analyzed by gel filtration. The affinity of the scFv′ monomer and (scFv′)₂ dimer can optionally be determined by BIAcore.

The (scFv′)₂ dimer may be created by joining the scFv fragments through a linker, e.g., through a peptide linker. This can be accomplished by a wide variety of means well known to those of skill in the art. For example, one suitable approach is described by Holliger et al. (1993) Proc. Natl. Acad. Sci. USA, 90:6444-6448 (see also WO 94/13804).

Typically, linkers are introduced by PCR cloning. For example, synthetic oligonucleotides encoding the 5 amino acid linker (Gly₄Ser, SEQ ID NO:22) can be used to PCR amplify the anti-CS3D antibody V_{H} and V_{L} genes which are then spliced together to create the anti-CS3D diabody gene. The gene can then be cloned into an appropriate vector, expressed, and purified according to standard methods well known to those of skill in the art.

Preparation of (scFv) 2, Fab, and (Fab′); Molecules

Antibodies such as scFv, or variant(s) with higher affinity, are suitable templates for creating size and valency variants. For example, an (scFv′)₂ can be created from the parent scFv as described above. An scFv gene can be excised using appropriate restriction enzymes and cloned into another vector as described herein.

Expressed scFv may include a myc tag at the C-terminus, followed by two glycines, a cysteine, and six histidines to facilitate purification. After disulfide bond formation between the two cystine residues, the two scFv may be separated from each other by 26 amino acids (e.g., two eleven amino acid myc tags and four glycines). Single-chain Fv (scFv) can be expressed from this construct and purified.

To produce (scFv′)₂ dimers, the cysteine is reduced by incubation with 1 mM β-mercaptoethanol, and half of the scFv blocked by the addition of DTNB. Blocked and unblocked scFv are incubated together to form (scFv′)₂, which is purified. As higher affinity scFv are isolated, their genes are similarly used to construct (scFv′)₂.

Fab may also be expressed in E. coli using an expression vector similar to the one described by Better et al. (1988) Science, 240:1041-1043. For example, to create a Fab, the V_{H} and V_{L} genes are amplified from the scFv using PCR. The V_{H} gene is cloned into an expression vector (e.g., a pUC119 based bacterial expression vector) that provides an IgG C_{H}1 domain downstream from, and in frame with, the V_{H} gene. The vector also contains the lac promoter, a pelB leader sequence to direct expressed V_{H}-C_{H}1 domain into the periplasm, a gene 3 leader sequence to direct expressed light chain into the periplasm, and cloning sites for the light chain gene. Clones containing the correct Vu gene are identified, e.g., by PCR fingerprinting. The V_{L} gene is spliced to the C_{L} gene using PCR and cloned into the vector containing the V_{H} C_{H}1 gene.

As indicated above, purification of the anti-CS3D antibody can be facilitated by cloning of the scFv gene into an expression vector (e.g., expression vector pUC119mycHIS) that results in the addition of the myc peptide tag followed by a hexa-histidine tag at the C-terminal end of the scFv. The vector may also encode a pectate lyase leader sequence that directs expression of the scFv into the bacterial periplasm where the leader sequence is cleaved. This makes it possible to harvest native properly folded scFv directly from the bacterial periplasm. The antibody is then expressed and purified from the bacterial supernatant using immobilized metal affinity chromatography.

### Measurement of Antibody Affinity

As explained above, selection for increased avidity involves measuring the affinity of an antibody for a target of interest. For example, the K_{D} of a antigen-binding antibody and the kinetics of binding to antigen are determined in a BIAcore, a biosensor based on surface plasmon resonance. For this technique, antigen is coupled to a derivatized sensor chip capable of detecting changes in mass. When antibody is passed over the sensor chip, antibody binds to the antigen resulting in an increase in mass that is quantifiable.

Measurement of the rate of association as a function of antibody concentration can be used to calculate the association rate constant (kₒₙ). After the association phase, buffer is passed over the chip and the rate of dissociation of antibody (k_{off}) determined. The equilibrium constant K_{d} is then calculated as k_{off}/kₒₙ and thus is typically measured in the range 10⁻⁷ to 10⁻¹² M. Affinities measured in this manner usually correlate well with affinities measured in solution by fluorescence quench titration.

V. Humanized, human engineered or human antibody production

The present antibodies and fragments can be humanized or human engineered antibodies. As used herein, a humanized antibody, or antigen binding fragment thereof, is a recombinant polypeptide that comprises a portion of an antigen binding site from a non-human antibody and a portion of the framework and/or constant regions of a human antibody. A human engineered antibody or antibody fragment may be derived from a human or non-human (e.g., mouse) source that has been engineered by modifying (e.g., deleting, inserting, or substituting) amino acids at specific positions so as to alter certain biophysical properties or to reduce any detectable immunogenicity of the modified antibody in a human.

Humanized antibodies also encompass chimeric antibodies and CDR-grafted antibodies in which various regions may be derived from different species. Chimeric antibodies may be antibodies that include a non-human antibody variable region linked to a human constant region. Thus, in chimeric antibodies, the variable region is mostly non-human, and the constant region is human. Chimeric antibodies and methods for making them are described in Morrison, et al., Proc. Natl. Acad. Sci. USA, 81:6841-6855 (1984), Boulianne, et al., Nature, 312:643-646 (1984), and PCT Application Publication WO 86/01533. Although, they can be less immunogenic than a mouse monoclonal antibody, administrations of chimeric antibodies have been associated with human anti-mouse antibody responses (HAMA) to the non-human portion of the antibodies. Chimeric antibodies can also be produced by splicing the genes from a mouse antibody molecule of appropriate antigen-binding specificity together with genes from a human antibody molecule of appropriate biological activity, such as the ability to activate human complement and mediate ADCC. Morrison et al. (1984), Proc. Natl. Acad. Sci., 81:6851; Neuberger et al. (1984), Nature, 312:604. One example is the replacement of an Fc region with that of a different isotype.

CDR-grafted antibodies are antibodies that include the CDRs from a non-human “donor” antibody linked to the framework region from a human “recipient” antibody. Generally, CDR-grafted antibodies include more human antibody sequences than chimeric antibodies because they include both constant region sequences and variable region (framework) sequences from human antibodies. Thus, for example, a CDR-grafted humanized antibody may comprise a heavy chain that comprises a contiguous amino acid sequence (e.g., about 5 or more, 10 or more, or even 15 or more contiguous amino acid residues) from the framework region of a human antibody (e.g., FR-1, FR-2, or FR-3 of a human antibody) or, optionally, most or all of the entire framework region of a human antibody. CDR-grafted antibodies and methods for making them are described in, Jones et al., Nature, 321:522-525 (1986), Riechmann et al., Nature, 332:323-327 (1988), and Verhoeyen et al., Science, 239:1534-1536 (1988)). Methods that can be used to produce humanized antibodies also are described in U.S. Pat. Nos. 4,816,567, 5,721,367, 5,837,243, and 6,180,377. CDR-grafted antibodies are considered less likely than chimeric antibodies to induce an immune reaction against non-human antibody portions. However, it has been reported that framework sequences from the donor antibodies are required for the binding affinity and/or specificity of the donor antibody, presumably because these framework sequences affect the folding of the antigen-binding portion of the donor antibody. Therefore, when donor, non-human CDR sequences are grafted onto unaltered human framework sequences, the resulting CDR-grafted antibody can exhibit, in some cases, loss of binding avidity relative to the original non-human donor antibody. See, e.g., Riechmann et al., Nature, 332:323-327 (1988), and Verhoeyen et al., Science, 239:1534-1536 (1988).

Human engineered antibodies include for example “vencered” antibodies and antibodies prepared using HUMAN ENGINEERING™ technology (U.S. Pat. No. 5,869,619). HUMAN ENGINEERING™ technology is commercially available, and involves altering an non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Techniques for making human engineered proteins are described in Studnicka ct al., Protein Engineering, 7:805-814 (1994), U.S. Pat. Nos. 5,766,886, 5,770,196, 5,821,123, and 5,869,619, and PCT Application Publication WO 93/11794.

“Veneered” antibodies are non-human or humanized (e.g., chimeric or CDR-grafted antibodies) antibodies that have been engineered to replace certain solvent-exposed amino acid residues so as to further reduce their immunogenicity or enhance their function. As surface residues of a chimeric antibody are presumed to be less likely to affect proper antibody folding and more likely to elicit an immune reaction, veneering of a chimeric antibody can include, for instance, identifying solvent-exposed residues in the non-human framework region of a chimeric antibody and replacing at least one of them with the corresponding surface residues from a human framework region. Veneering can be accomplished by any suitable engineering technique, including the use of the above-described HUMAN ENGINEERING™ technology.

In a different approach, a recovery of binding avidity can be achieved by “de-humanizing” a CDR-grafted antibody. De-humanizing can include restoring residues from the donor antibody's framework regions to the CDR grafted antibody, thereby restoring proper folding. Similar “de-humanization” can be achieved by (i) including portions of the “donor” framework region in the “recipient” antibody or (ii) grafting portions of the “donor” antibody framework region into the recipient antibody (along with the grafted donor CDRs).

For a further discussion of antibodies, humanized antibodies, human engineered, and methods for their preparation, see Kontermann and Dubel, eds., Antibody Engineering, Springer, New York, NY, 2001.

The present antibodies and fragments encompass antibodies having CDRs of human origin, such as antibodies which bind the antigen and are encoded by nucleic acid sequences which are naturally occurring somatic variants of human germline immunoglobulin nucleic acid sequence, and fragments, synthetic variants, derivatives and fusions thereof. Such antibodies may be produced by any method known in the art, such as through the use of transgenic mammals (such as transgenic mice) in which the native immunoglobulin repertoire has been replaced with human V-genes in the mammal chromosome. Such mammals appear to carry out VDJ recombination and somatic hypermutation of the human germline antibody genes in a normal fashion, thus producing high affinity antibodies with completely human sequences.

Human antibodies can also be generated through the in vitro screening of antibody display libraries. See Hoogenboom et al. (1991), J. Mol. Biol. 227:381; and Marks et al. (1991), J. Mol. Biol. 222:581. Various antibody-containing phage display libraries have been described and may be readily prepared. Libraries may contain a diversity of human antibody sequences, such as human Fab, Fv, and scFv fragments that may be screened against an appropriate target. Phage display libraries may comprise peptides or proteins other than antibodies which may be screened to identify selective binding agents.

### VI. Other Antibody Forms

Sequence provided herein can be used to generate other antibody forms, including but not limited to nanobodies, UniBodies, and/or affibodies.

VHH and/or Nanobodies

The Camelidae heavy chain antibodies are found as homodimers of a single heavy chain, dimerized via their constant regions. The variable domains of these camelidae heavy chain antibodies are referred to as VHH domains or VHH, and can be either used per se as nanobodies and/or as a starting point for obtaining nanobodies. Isolated VHH retain the ability to bind antigen with high specificity (see, e.g., Hamers-Casterman et al. (1993) Nature 363:446-448). VHH domains, or nucleotide sequences encoding them, can be derived from antibodies raised in Camelidae species, for example in camel, dromedary, llama, alpaca and guanaco. Other species besides Camelidae (e.g, shark, pufferfish) can produce functional antigen-binding heavy chain antibodies, from which (nucleotide sequences encoding) such naturally occurring VHH can be obtained, e.g., using the methods described in U.S. Patent Publication US 2006/0211088.

Human proteins may be used in therapy primarily because they are not as likely to provoke an immune response when administered to a patient. Comparisons of camelid VHH with the V_{H} domains of human antibodies reveals several key differences in the framework regions of the camelid VHH domain corresponding to the V_{H}/V_{L} interface of the human V_{H} domains. Mutation of these human residues to VHH resembling residues has been performed to produce “camelized” human VH domains that retain antigen binding activity, yet have improved expression and solubility.

Libraries of single V_{H} domains have also been derived for example from VH genes amplified from genomic DNA or from mRNA came from the spleens of immunized mice and expressed in E. coli (Ward et al. (1989) Nature 341:544-546) and similar approaches can be performed using the V_{H} domains and/or the V_{L} domains described herein. The isolated single V_{H} domains are called “dAbs” or domain antibodies. A “dAb” is an antibody single variable domain (V_{H} or V_{L}) polypeptide that specifically binds antigen. A “dAb” binds antigen independently of other V domains; however, as the term is used herein, a “dAb” can be present in a homo- or heteromultimer with other V_{H} or V_{L} domains where the other domains are not required for antigen binding by the dAb, i.e., where the dAb binds antigen independently of the additional VH or VL domains.

As described in U.S. Patent Publication No. 2006/0211088 methods are known for the cloning and direct screening of immunoglobulin sequences (including but not limited to multivalent polypeptides comprising: two or more variable domains-—or antigen binding domains-—and in particular VH domains or VHH domains; fragments of VL, VH or VHH domains, such as CDR regions, for example CDR3 regions; antigen-binding fragments of conventional 4-chain antibodies such as Fab fragments and scFv's, heavy chain antibodies and domain antibodies; and in particular of VH sequences, and more in particular of VHH sequences) that can be used as part of and/or to construct such nanobodies.

Methods and procedures for the production of VHH/nanobodies can also be found for example in WO 94/04678, WO 96/34103, WO 97/49805, WO 97/49805 WO 94/25591, WO 00/43507 WO 01/90190, WO 03/025020, WO 04/062551, WO 04/041863, WO 04/041865, WO 04/041862, WO 04/041867, PCT/BE2004/000159, Hamers-Casterman et al. (1993) Nature 363:446; Riechmann and Muyldermans (1999) J. Immunological Meth., 231:25-38; Vu et al. (1997) Molecular Immunology, 34 (16-17): 1121-1131; Nguyen et al. (2000) EMBO J., 19 (5): 921-930; Arbabi Ghahroudi et al. (19997) FEBS Letters 414:521-526; van der Linden et al. (2000) J. Immunological Meth., 240:185-195; Muyldermans (2001) Rev. Molecular Biotechnology 74:277-302; Nguyen el al. (2001) Adv. Immunol. 79:261, and the like, which are all incorporated herein by reference.

### UniBodies

UniBodies are generated by an antibody technology that produces a stable, smaller antibody format with an anticipated longer therapeutic window than certain small antibody formats. UniBodies may be produced from IgG4 antibodies by eliminating the hinge region of the antibody. Unlike the full size IgG4 antibody, the half molecule fragment is very stable and is termed a UniBody. Halving the IgG4 molecule left only one area on the UniBody that can bind to a target. Methods of producing UniBodies are described in detail in PCT Publication WO2007/059782, which is incorporated herein by reference in its entirety (see, also, Kolfschoten et al. (2007) Science 317:1554-1557).

### Affibodies

Affibody molecules are class of affinity proteins based on a 58-amino acid residue protein domain, derived from one of the IgG-binding domains of staphylococcal protein A. This three-helix bundle domain has been used as a scaffold for the construction of combinatorial phagemid libraries, from which affibody variants that target the desired molecules can be selected using phage display technology (see, e.g., Nord et al. (1997) Nat. Biotechnol. 15:772-777; Ronmark et al. (2002) Eur. J. Biochem., 269:2647-2655.). Details of affibodies and methods of production are known to those of skill (see, e.g., U.S. Pat. No. 5,831,012 which is incorporated herein by reference in its entirety).

### IX. Assays

As explained above, the antibodies of the present disclosure can be used for the in vivo or in vitro detection of CS3D and thus, are useful in the measuring pharmacokinetics of CS3D (e.g., in a human subject) and/or visualization of CS3D in vivo or in vitro.

The CS3D can be quantified in a biological sample derived from a subject that has been administered CS3D (e.g., a human patient, a mouse model, etc.). As used herein, a biological sample is a sample of biological tissue or fluid that contains CS3D. Examples of suitable biological samples include blood (or blood fraction such as serum or plasma), brain interstitial fluid (ISF), cerebrospinal fluid, urine, saliva, tissue biopsies, etc.

Although the sample is typically taken from a human patient, the assays can be used to detect CS3D in samples from mammals in general, such as dogs, cats, sheep, cattle and pigs, and most particularly primates such as humans, chimpanzees, gorillas, macaques, and baboons, and rodents such as mice, rats, and guinea pigs.

Tissue or fluid samples are isolated from a patient according to standard methods well known to those of skill in the art, most typically by biopsy or venipuncture. The sample is optionally pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

### Pharmacokinetic Assays

The pharmacokinetics of CS3D may be examined in a subject (e.g., humans) following a single intravenous IV injection (or another route of administration) at multiple dose levels. Doses for IV administration may be 1, 5, and 15 mg/kg. Blood samples may be collected at selected time points over a time course. The resultant serum samples can be analyzed using an ELISA assay for total CS3D concentrations. Pharmacokinetic (PK) parameter estimates such as observed maximal concentration in serum (Cmax), the time of observed maximal concentration (Tmax), area under the concentration vs. time curve (AUC), clearance (CL), volume of distribution (Vz), and mean residence time (MRT) may be determined. The bioavailability following IV administration may be determined. Half-life may also be determined.

### Immunological Binding Assays

CS3D can be detected in an immunoassay utilizing the antibodies of the present disclosure as a capture agent that specifically binds to the CS3D.

As used herein, an immunoassay is an assay that utilizes an antibody to specifically bind an analyte. The immunoassay is characterized by the binding of an antibody to a target as opposed to other physical or chemical properties to isolate, target, and quantify the CS3D.

CS3D can be detected and quantified using any of a number of well recognized immunological binding assays. For example, the antibody of the present disclosure may be immobilized on a substrate (e.g., bead) and/or be the capture antibody in an ELISA. The detection step may take one of many formats known in the art, such as using a labeled secondary antibody or PCR amplification. Where PCR amplification is the method of detection, the antibody is conjugated to a nucleic acid, the antigen may optionally be first attached to a substrate, and the antibody is allowed to be bound to the antigen. The bound antibody-nucleic acid fusion then undergoes PCR amplification of the nucleic acid sequence attached to the antibody. The amplified sequences can in turn be detected via a fluorophore bound to the incorporated nucleotides. The amplified sequences can also be first hybridized to an array before fluorescence is measured to enable multiplexing. Multiplexing encompasses processing and detecting two or more samples and/or two or more analytes in parallel. Details of an assay using antibody-nucleic acid fusion may be found in US20060141505, disclosure of which is incorporated by reference.

Single assay or multiplex assay can also take the form of an array where signal is detected only by electro-stimulation. In this format, the antibody of the present disclosure is conjugated to an electrochemiluminescent moiety and immobilized on an electrode. A signal (e.g., fluorescence) is emitted due to electrical stimulation at a particular electrode. Details of an assay using electrochemiluminescent moiety in an array may be found in US20100140086, disclosure of which is incorporated by reference.

A fluorescent compound may be also added later to the assay for visualization by either Luminex type or other type of detection (see, e.g., U.S. Pat. Nos. 4,366,241; 4,376,110; 4,517,288; and 4,837,168, and the like). For a review of the general immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991)).

The immunoassays of the present disclosure can be performed in any of a number of configurations (see, e.g., those reviewed in Maggio (ed.) (1980) Enzyme Immunoassay CRC Press, Boca Raton, Florida; Tijan (1985) “Practice and Theory of Enzyme Immunoassays,” Laboratory Techniques in Biochemistry and Molecular Biology, Elsevier Science Publishers B.V., Amsterdam; Harlow and Lane, supra; Chan (ed.) (1987) Immunoassay: A Practical Guide Academic Press, Orlando, FL; Price and Newman (eds.) (1991) Principles and Practice of Immunoassays Stockton Press, NY; and Ngo (ed.) (1988) Non isotopic Immunoassays Plenum Press, NY).

Immunoassays often utilize a labeling agent to specifically bind to and label the binding complex formed by the capture agent and the antigen (e.g., an anti-CS3D antibody and CS3D complex). The labeling agent can itself be one of the moieties comprising the antibody/analyte complex. Thus, for example, the labeling agent can be a labeled anti-CS3D antibody. Alternatively, the labeling agent is optionally a third moiety, such as another antibody, that specifically binds to the CS3D antibody, the CS3D, the antibody/CS3D complex, or to a modified capture group (e.g., biotin) which is covalently linked to CS3D or to the anti-CS3D antibody.

The labeling agent encompasses an antibody that specifically binds to the anti-CS3D antibody. Such agents are well known to those of skill in the art, and most typically comprise labeled antibodies that specifically bind antibodies of the particular animal species from which the anti-CS3D antibody is derived (e.g., an anti-species antibody). Thus, for example, where the capture agent is a human derived CS3D antibody, the label agent may be a mouse anti-human IgG, i.e., an antibody specific to the constant region of the human antibody.

Other proteins capable of specifically binding immunoglobulin constant regions, such as streptococcal protein A or protein G are also used as the labeling agent. These proteins are normal constituents of the cell walls of streptococcal bacteria. They exhibit a strong non-immunogenic reactivity with immunoglobulin constant regions from a variety of species (see generally Kronval, et al., (1973) J. Immunol., 111:1401-1406, and Akerstrom, et al., (1985) J. Immunol., 135:2589-2542, and the like).

Throughout the assays, incubation and/or washing steps may be required after each combination of reagents. Incubation steps can vary from about 5 seconds to several hours, for example, from about 5 minutes to about 24 hours (e.g., from 5 minutes to 15 minutes, from 15 minutes to 30 minutes, from 30 minutes to 60 minutes, from 1 hour to 4 hours, from 4 hours to 8 hours, from 8 hours to 12 hours, or from 12 hours to 24 hours). However, the incubation time will depend upon the assay format, analyte, volume of solution, concentrations, and the like. Usually, the assays are carried out at ambient temperature, although they can be conducted over a range of temperatures, such as 5° C. to 45° C.

### Noncompetitive Assay Formats

Immunoassays for detecting CS3D may be either competitive or noncompetitive. Noncompetitive immunoassays are assays in which the amount of captured analyte (in this case, CS3D) is directly measured. In one example of a suitable “sandwich” assay, the capture agent (e.g., an CS3D antibody) is bound directly or indirectly to a solid substrate where it is immobilized. These immobilized anti-CS3D antibodies capture CS3D present in a test sample (e.g., a blood sample). The CS3D thus immobilized are then bound by a labeling agent, e.g., an anti-CS3D antibody bearing a label. Alternatively, the second antibody may lack a label, but it may, in turn, be bound by a labeled third antibody specific to antibodies of the species from which the second antibody is derived. Free labeled antibody is washed away and the remaining bound labeled antibody is detected (e.g., using a gamma detector where the label is radioactive).

### Competitive Assay Formats

In competitive assays, the amount of CS3D present in the sample is measured indirectly by measuring the amount of an added CS3D displaced (or competed away) from a capture agent (e.g., anti-CS3D antibody) by the CS3D present in the sample. For example, in one competitive assay, a known amount of CS3D is added to a test sample with an unquantified amount of CS3D, and the sample is contacted with a capture agent, e.g., an CS3D antibody that specifically binds CS3D. The amount of added CS3D that binds to the anti-CS3D antibody is inversely proportional to the concentration of CS3D present in the test sample.

The anti-CS3D antibody can be immobilized on a solid substrate. The amount of CS3D bound to the anti-CS3D antibody is determined either by measuring the amount of CS3D present in a CS3D-anti-CS3D antibody complex, or alternatively by measuring the amount of remaining uncomplexed CS3D.

### Reduction of Non-Specific Binding

One of skill will appreciate that it is often desirable to reduce non-specific binding in immunoassays and during CS3D purification. Where the assay involves, for example CS3D, CS3D-binding antibody, or other capture agent(s) immobilized on a solid substrate, it is desirable to minimize the amount of non-specific binding to the substrate. Means of reducing such non-specific binding are well known to those of skill in the art. Typically, this involves coating the substrate with a proteinaceous composition. In particular, protein compositions such as bovine serum albumin (BSA), nonfat powdered milk, and gelatin are widely used.

### Substrates

As mentioned above, depending upon the assay, various components are optionally bound to a solid surface. Many methods for immobilizing biomolecules to a variety of solid surfaces are known in the art. For instance, the solid surface may be a membrane (e.g., nitrocellulose), a microtiter dish (e.g., PVC, polypropylene, or polystyrene), a test tube (glass or plastic), a dipstick (e.g., glass, PVC, polypropylene, polystyrene, latex, and the like), a microcentrifuge tube, or a glass, silica, plastic, metallic or polymer bead. The desired component may be covalently bound, or noncovalently attached through nonspecific bonding.

A wide variety of organic and inorganic polymers, both natural and synthetic may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials which may be employed include paper, glasses, ceramics, metals, metalloids, semiconductive materials, cements or the like. In addition, substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes may be employed depending upon the nature of the system.

In preparing the surface, a plurality of different materials may be employed, e.g., as laminates, to obtain various properties. For example, protein coatings, such as gelatin can be used to avoid non-specific binding, simplify covalent conjugation, and enhance signal detection or the like.

If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. See, for example, Immobilized Enzymes, Ichiro Chibata, Halsted Press, New York, 1978, and Cuatrecasas, (1970) J. Biol. Chem. 245 3059.

In addition to covalent bonding, various methods for noncovalently binding an assay component can be used. Noncovalent binding is typically nonspecific absorption of a compound to the surface. Typically, the surface is blocked with a second compound to prevent nonspecific binding of labeled assay components. Alternatively, the surface is designed such that it nonspecifically binds one component but does not significantly bind another. For example, a surface bearing a lectin such as concanavalin A will bind a carbohydrate containing compound but not a labeled protein that lacks glycosylation. Various solid surfaces for use in noncovalent attachment of assay components are reviewed in U.S. Pat. Nos. 4,447,576 and 4,254,082, which is incorporated herein by reference.

### Labeling of Anti-CS3D Antibodies

Anti-CS3D antibodies can be labeled by any of a number of methods known to those of skill in the art. Thus, for example, the labeling agent can be, e.g., a monoclonal antibody, a polyclonal antibody, a protein or complex such as those described herein, or a polymer such as an affinity matrix, carbohydrate or lipid. Detection proceeds by any known method, including immunoblotting, western analysis, gel-mobility shift assays, tracking of radioactive or bioluminescent markers, nuclear magnetic resonance, electron paramagnetic resonance, stopped-flow spectroscopy, column chromatography, capillary electrophoresis, or other methods which track a molecule based upon an alteration in size and/or charge. The detectable group can be any material having a detectable physical or chemical property. Such detectable labels have been well-developed in the field of immunoassays and, in general, any label useful in such methods can be applied in the various embodiments of the present disclosure. Thus, a label is any composition detectable by spectroscopic, photochemical, biochemical, immunochemical, electrical, optical or chemical means. Useful labels in the present disclosure include magnetic beads (e.g. Dynabeads™), fluorescent dyes (e.g., fluorescein isothiocyanate, Texas red, rhodamine, Alexa fluor dyes and the like), radiolabels (e.g., ³H, ¹²⁵I, ³⁵S, ¹⁴C, or ³²P), enzymes (e.g., LacZ, CAT, horse radish peroxidase, luciferase, alkaline phosphatase and others, commonly used as detectable enzymes, either as marker gene products or in an ELISA), and colorimetric labels such as colloidal gold or colored glass or plastic (e.g. polystyrene, polypropylene, latex, etc.) beads. For example, an antibody can include a fluorescent label, a chemiluminescent label, a radiolabel, a chromogenic label, or other suitable label.

The label may be coupled directly or indirectly to the desired component of the assay according to methods well known in the art. As indicated above, a wide variety of labels may be used, with the choice of label depending on the sensitivity required, case of conjugation of the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904, which is incorporated herein by reference.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

The anti-CS3D antibodies provided by the present disclosure are useful for parenteral, topical, oral, or local administration for visualizing CS3D in the body of a subject who has been administered. A pharmaceutical composition comprising the antibodies of the present disclosure can be administered in a variety of unit dosage forms depending upon the method of administration.

The pharmaceutical compositions of the present disclosure may include an antibody as provided herein suspended in a pharmaceutically acceptable carrier, which may be an aqueous carrier. A variety of aqueous carriers can be used, e.g., buffered saline and the like.

### XI. Kits for Diagnosis or Treatment

Kits for the detection of CS3D are also provided. Kits will typically comprise one or more anti-CS3D antibodies. The antibody(s) can optionally be labeled. In addition, the kits will typically include instructional materials disclosing means of use anti-CS3D antibodies in the detection of CS3D. The kits may also include additional components to facilitate the particular application for which the kit is designed. The kit can additionally contain means of detecting the label (e.g., enzyme substrates for enzymatic labels, filter sets to detect fluorescent labels, appropriate secondary labels such as a sheep anti-human antibody, or the like). The kits may additionally include buffers and other reagents routinely used for the practice of a particular method. Such kits and appropriate contents are well known to those of skill in the art.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-39 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. An isolated antibody that specifically binds to a cyclic STAT3 double-stranded oligonucleotide decoy (CS3D).
2. An isolated antibody that specifically binds to a cyclic STAT3 double-stranded oligonucleotide decoy (CS3D) and competes for binding to the CS3D with
(i) a first antibody comprising:
   a variable heavy (VH) chain comprising heavy chain complementarity determining regions 1-3 (HCDRs 1-3) of a VH chain comprising the sequence:

(SEQ ID NO: 1)
QVQLQESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKGL
RYSSGWGQGTMVTVSS;

and
a variable light (VL) chain comprising light chain complementarity determining regions 1-3 (LCDRs 1-3) of a VL chain comprising the sequence:

(SEQ ID NO: 2)
QPVLTQPPSVSKGLRQTATLTCSGNSNNVGNAGAVWLQQHQGHPPKLLSY
RNNNRPSGISERISASRSGNTASLTISGLQPEDEADYYCSAWDSSLRVQV
FGGGTKLTVL;

or
(ii) a second antibody comprising:
   a VH chain comprising HCDRs 1-3 of a VH chain comprising the sequence:

(SEQ ID NO: 3)
QVTLQQSGPGLVKPSQTLSLTCAISGDSVSSNSAAWNWIRQSPSRGLEWL
GRTYYRSKWFTYYAASVKGRISINRDTSKNQFSLQLNSVTPDDTAVYFCA
RGSGGYIDHWGQGTLVTVSS;

and
a VL comprising LCDRs 1-3 of a VL chain comprising the sequence:

(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

3. An isolated antibody that specifically binds to a cyclic STAT3 double-stranded oligonucleotide decoy (CS3D) and comprises:
a variable heavy (VH) chain comprising heavy chain complementarity determining regions 1-3 (HCDRs 1-3) of a VH chain comprising the sequence:

(SEQ ID NO: 1)
QVQLQESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKGL
RYSSGWGQGTMVTVSS;

and
a variable light (VL) chain comprising light chain complementarity determining regions 1-3 (LCDRs 1-3) of a VL chain comprising the sequence:

(SEQ ID NO: 2)
QPVLTQPPSVSKGLRQTATLTCSGNSNNVGNAGAVWLQQHQGHPPKLLSY
RNNNRPSGISERISASRSGNTASLTISGLQPEDEADYYCSAWDSSLRVQV
FGGGTKLTVL.

4. An isolated antibody that specifically binds to a cyclic STAT3 double-stranded oligonucleotide decoy (CS3D) and comprises:
   a variable heavy (VH) chain comprising heavy chain complementarity determining regions 1-3 (HCDRs 1-3) of a VH chain comprising the sequence:

(SEQ ID NO: 3)
QVTLQQSGPGLVKPSQTLSLTCAISGDSVSSNSAAWNWIRQSPSRGLEWL
GRTYYRSKWFTYYAASVKGRISINRDTSKNQFSLQLNSVTPDDTAVYFCA
RGSGGYIDHWGQGTLVTVSS;

and
a variable light (VL) chain comprising light chain complementarity determining regions 1-3 (LCDRs 1-3) of a VL chain comprising the sequence:

(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

5. The isolated antibody of any one of aspects 1-4, wherein the antibody comprises:
a VH chain comprising a HCDR1 comprising the amino acid sequence GFTFSSYAMS (SEQ ID NO: 5), a HCDR2 comprising the amino acid sequence SAISGSGGSTY (SEQ ID NO:6), and a HCDR3 comprising the amino acid sequence GLRYSSG (SEQ ID NO:7), and
wherein the VH chain comprises an amino acid sequence at least 85% identical, at least 90% identical, at least 95% identical, at least 96% identical, at least 97% identical, or at least 98% identical to the amino acid sequence of SEQ ID NO:1.

6. The isolated antibody of any one of aspects 1-5, wherein the antibody comprises:
a VL chain comprising a LCDR1 comprising the amino acid sequence SGNSNNVGNAGAV (SEQ ID NO:8), a LCDR2 comprising the amino acid sequence NNNRPS (SEQ ID NO:9), and a LCDR3 comprising the amino acid sequence SAWDSSLRVQV (SEQ ID NO:10), and
wherein the VL chain comprises an amino acid sequence at least 85% identical, at least 90% identical, at least 95% identical, at least 96% identical, at least 97% identical, or at least 98% identical to the amino acid sequence of SEQ ID NO:2.

7. The isolated antibody of any one of aspects 1-4, wherein the antibody comprises:
a VH chain comprising a HCDR1 comprising the amino acid sequence GDSVSSNSAAWN (SEQ ID NO: 11), a HCDR2 comprising the amino acid sequence GRTYYRSKWFTY (SEQ ID NO:12), and a HCDR3 comprising the amino acid sequence GSGGYIDH (SEQ ID NO:13), and
wherein the VH chain comprises an amino acid sequence at least 85% identical, at least 90% identical, at least 95% identical, at least 96% identical, at least 97% identical, or at least 98% identical to the amino acid sequence of SEQ ID NO:3.
8. The isolated antibody of aspect 7, wherein the antibody comprises:
a VL chain comprising a LCDR1 comprising the amino acid sequence TLRSGINVDSYRIY (SEQ ID NO:14), a LCDR2 comprising the amino acid sequence DKQQGS (SEQ ID NO:15), and a LCDR3 comprising the amino acid sequence MIWHSSAVV (SEQ ID NO:16), and
wherein the VL chain comprises an amino acid sequence at least 85% identical, at least 90% identical, at least 95% identical, at least 96% identical, at least 97% identical, or at least 98% identical to the amino acid sequence of SEQ ID NO:4.
9. The isolated antibody of any one of aspects 1-8, wherein the antibody is a single chain Fv (scFv), IgG, Fab, (Fab) 2, or (scFv) 2.
10. The isolated antibody of any one of aspects 1-9, wherein the antibody is a human antibody or a humanized antibody.
11. The isolated antibody of any one of aspects 1-10, wherein the antibody is labeled.
12. The isolated antibody of any one of aspects 1-10, wherein the antibody is attached to a solid support.
13. An isolated nucleic acid comprising a nucleotide sequence encoding the VH chain and the VL chain of the antibody of any one of aspects 1-10.
14. A vector comprising the isolated nucleic acid of aspect 13.
15. A cell comprising the nucleic acid of aspect 13 or the vector of aspect 14.
16. A composition comprising a first nucleic acid comprising a nucleotide sequence encoding the VH chain and a second nucleic acid comprising a nucleotide sequence encoding the VL chain of the antibody of any one of aspects 1-10.
17. A cell comprising the first and second nucleic acids of aspect 61.
18. A composition comprising a first vector comprising a nucleotide sequence encoding the VH chain and a second vector comprising a nucleotide sequence encoding the VL chain of the antibody of any one of aspects 1-10.
19. A cell comprising the first and second vectors of aspect 18.
20. A method for detecting a cyclic STAT3 double-stranded oligonucleotide decoy (CS3D) in a sample, comprising:
   contacting an antibody of any one of aspects 1-10 with the sample,
   detecting binding of the antibody to CS3D in the sample.
21. The method of aspect 20, wherein the antibody is labeled.
22. The method of aspect 21, wherein the label is a fluorescent label, a chemiluminescent label, a radiolabel, an enzyme, or a chromogenic label.
23. The method of any one of aspects 20-22, wherein the sample is blood, serum, or plasma.
24. The method of any one of aspects 20-23, wherein the antibody is immobilized on a substrate.
25. The method of any one of aspects 20-24, further comprising determining a concetration of CS3D.
26. A method for detecting presence of detecting a cyclic STAT3 double-stranded oligonucleotide decoy (CS3D) in vivo in a subject, the method comprising:
administering the antibody of any one of aspects 1-10 to the subject; and
detecting the antibody in the subject,
wherein the subject has been administered the CS3D.

### Examples

The following examples are offered to illustrate, but not to limit any embodiments provided by the present disclosure.

### Overview

In vitro biopanning platforms using synthetic phage display Fab libraries have enabled the identification of antibodies against antigens that were once thought to be beyond the scope of immunization. Expanding these methods to identifying rare high-affinity binders against challenging targets remains a critical challenge. Here, we present a new biopanning pipeline, RAPID (Rare Antibody Phage Isolation and Discrimination), for the identification of rare high-affinity antibodies against challenging targets. RAPID biopanning uses fluorescent labeled phage displayed Fab libraries for the isolation of high-affinity binders with fluorescent activated sorting. Subsequently, discriminatory hit screening is performed with a newly developed biolayer interferometry (BLI) method, BIAS (Biolayer Interferometry Antibody Screen), where candidate binders are ranked and prioritized according to their estimated kinetic off rates. A well characterized antibody-antigen pair, P1A4 and ARS1620, were used to establish the pipeline, and two challenging targets (CS3D, and CH1P), were employed, where RAPID biopanning enabled for the identification of high-affinity antibodies.

Antibodies (Abs) have and continue to be one of the most invaluable tools for biological research¹. In particular, the utility of rapidly generated high-quality Abs has been highlighted in the 2020 SARS-COV-2 pandemic as attractive therapeutic interventions^{2,3,4}. While immunization has been the traditional method for Ab discovery, biopanning using synthetically displayed Ab libraries (i.e., phage display and yeast surface display) have expanded the field and allowed for non-protein antigen (Ag) targets such as DNA5 and RNA6 to be targeted.

Standard phage display biopanning methods largely consist of two stages: (1) Iterative rounds of washing and amplification of displayed Abs bound to immobilized Ag to achieve an enriched population of higher-affinity binders, and (2) Random screening of enriched pools of displayed Abs for the identification of high-affinity Ab clones (hits). Notably, the in vitro selection and screening nature of the method enables the identification of high-affinity Abs in conditions otherwise impossible with immunization (i.e., changes in pH, temperature, oligomeric complex states, ligand states etc.),

Despite these attractive features of biopanning, it remains challenging to identify, and characterize selective, high-affinity Abs where their prevalence's are extremely low (i.e., target Ag exhibits low antigenicity, and/or conformational heterogeneity). Poor enrichment towards Ag-Ab binding in turn allows additional factors (i.e., amplification discrepancies of phagemid-containing E. coli and inconsistent display propensities of phage) to undermine the enrichment process resulting in an overall depletion of promising binders with continued rounds of selection. In turn, significantly larger number of clones need to be screened and prioritized, and as this process largely relies on stochastic clone picking, the vast majority of candidate clones, and potentially rare high-affinity binders can be left unexamined.

These challenges have been met with some effective solutions such as improvements in synthetic Ab library design⁷, selective isolation of high-affinity binders using fluorescence-activated cell sorting (FACS)⁸ coupled with yeast surface display (YSD^{9,10}), and selection-based methods for identifying candidate clones¹¹. Yet, further improvements in the current platforms for efficient recombinant Ab discovery are crucial for continued expansion of the field.

Here we provide a novel phage display pipeline, RAPID (Rare Antibody Phage Isolation and Discrimination), for identifying rare high-affinity Abs against challenging targets using a recombinant phage displayed Fab (ph-Fab) library (FIG. 1). The RAPID biopanning pipeline was strategized to selectively isolate the most promising subset population of binders and subsequently perform screening in a discriminatory fashion allowing for a highly efficient strategy for the identification of high-affinity Fabs. This is distinct from the standard biopanning pipeline where the total population of enriched displayed Abs are non-discriminatorily screened. RAPID biopanning utilizes fluorescent labeling of ph-Fab libraries to precisely identify and isolate populations of rare high-affinity ph-Fabs, and a newly developed BLI (Biolayer interferometry) method called BIAS (Biolayer Interferometry Antibody Screen) is used for discriminatory screening of candidate clones. (FIG. 1).

RAPID biopanning combines the advantages of phage display by utilizing large library sizes (up to 10¹⁰) while leveraging the quantitative Ab-Ag binding screening afforded by the FACS methodology commonly used with YSD13. In addition, RAPID is not material intensive (~20 μg of Ag required) and therefore can be used for targets that are difficult to acquire reagent quantities of. Overall, the RAPID pipeline is widely applicable and allows for a highly efficient strategy for the identification of rare high-affinity binders.

Here, we first provide controls for each step of RAPID using a well characterized antibody, P1A4, which binds ARS1620, a preclinical, covalent KRas G12C inhibitor. Subsequently, two challenging biological panning campaigns are described as examples to highlight the power of RAPID biopanning. Cyclic STAT3 Decoy (CS3D) is a novel double stranded DNA drug candidate, and there is limited precedence for the description of Fabs that bind to dsDNA antigens. Carboxyl terminus of Hsp-70 interacting protein (CH1P) is a E3 ubiquitin ligase target that is highly conformationally diverse and for which previous in-house standard panning protocols failed to identify binders against.

### Example 1: The Rare Antibody Phage Isolation and Discrimination (RAPID) Pipeline

The RAPID biopanning pipeline comprises of four steps: (1) Fluorescent labeling of ph-Fab libraries for the detection and quantification of ph-Fab-Ag-bead complexes (2) Biopanning campaign enrichment progression profiling with flow cytometry for the accurate determination of Ag-Fab enrichment, (3) Isolation of high-affinity binders with fluorescence activated sorting, and (4) Discriminatory hit screening of candidate clones with BIAS (FIG. 1).

NHS-FITC Labeling of pH-Fab Enables Quantitative Detection of Fab-Ag Binding

For the RAPID pipeline to be a feasible protocol, fluorescence from Ag-Bead-phage complexes need to directly correlate to the number of ph-Fabs bound per Ag-Bead. In addition, labeling should occur with minimal variability between different ph-Fabs and not disrupt the interactions between displayed Fabs and bead immobilized Ags, so as to not introduce additional biases.

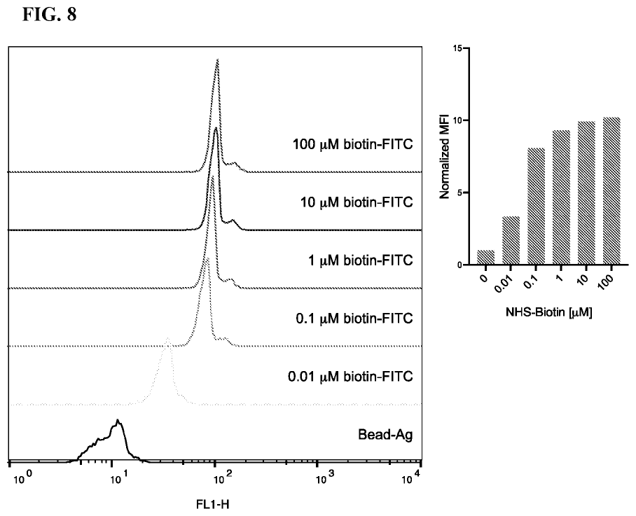

To define the dynamic range (or upper limits) of fluorescent labeling on beads, increasing concentration of biotin-fluorescein isothiocyanate (FITC) was immobilized to streptavidin beads and analyzed with flow cytometry (FIG. 8). Results indicate a concentration dependent shift of fluorescent signal distribution, which fully saturates by 1 mM, indicating that no self-quenching occurs from proximally immobilized FITC molecules at the upper limits of fluorescent signal.

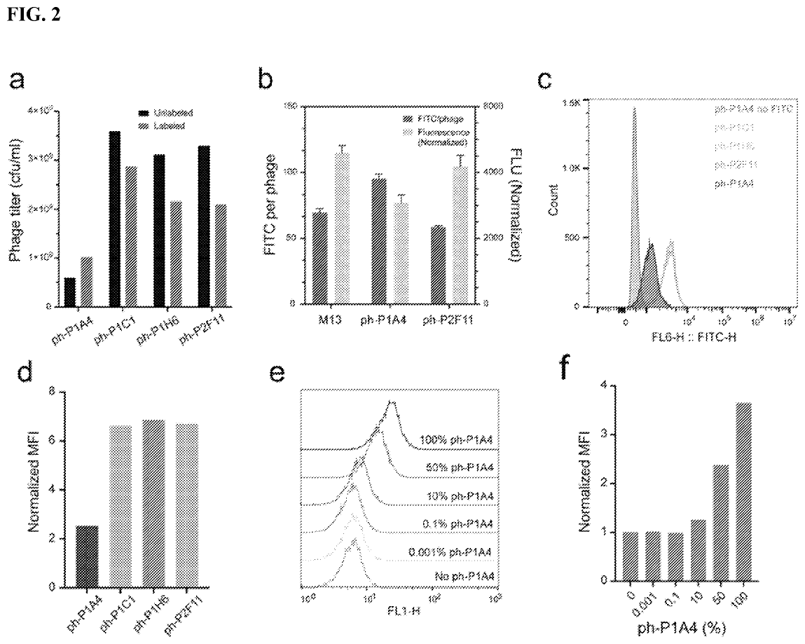

Four unique ph-Fabs that bind to the same Ag, ARS1620 (ph-P1A4, ph-P1C1, ph-P1H6, and ph-P2F11) were chosen to test if FITC labeling showed any significant variability between different ph-Fabs or if labeling affected ph-Fab binding to bead immobilized Ag. Pull down experiments of different ph-Fabs against the same Ag (ARS1620 peptide) show similar titers of bound phage between labeled and unlabeled phage, indicating that FITC labeling does not significantly disrupt displayed Fab-Ag binding (FIG. 2a). Comparing specific labeling parameters, ph-P1A4 and ph-P2F11 showed variations in both degrees of labeling (60-100 FITC per phage) and normalized fluorescence (~3000-4500 RFU) (FIG. 2b). However, given the pull-down titer ratio of labeled ph-P1A4 versus labeled ph-P2F11 (2.05-fold) roughly match that of the FITC median fluorescence intensity (MFI) ratio between the two ph-Fabs (2.64-fold), these variations can be considered inconsequential (FIG. 2a, 2c, 2d).

### RAPID Flow Cytometry for Profiling the Enrichment of a Biopanning Campaign

M13 phage and ph-P1A4 were mixed in different ratios to mimic different phage libraries with different hit rates. These mixtures were then individually FITC labeled, and flow cytometry analysis was performed with ARS1620 bound beads. A normalized MFI (to the 0% ph-P1A4 sample) of 1.26 is exhibited at 10% ph-P1A4 (FIG. 2e, 2f). Normalized MFI values for a 50% and 100% show 2.38 and 3.65 respectively which can act as a standard in determining future approximate hit rate of a library of ph-Fab with a k_{off} of 2.58 ×10⁻³ (1/s) (Kd~25 nM).

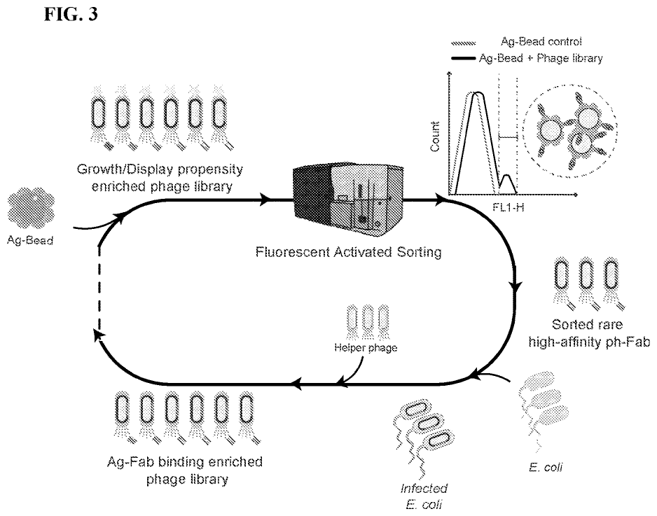

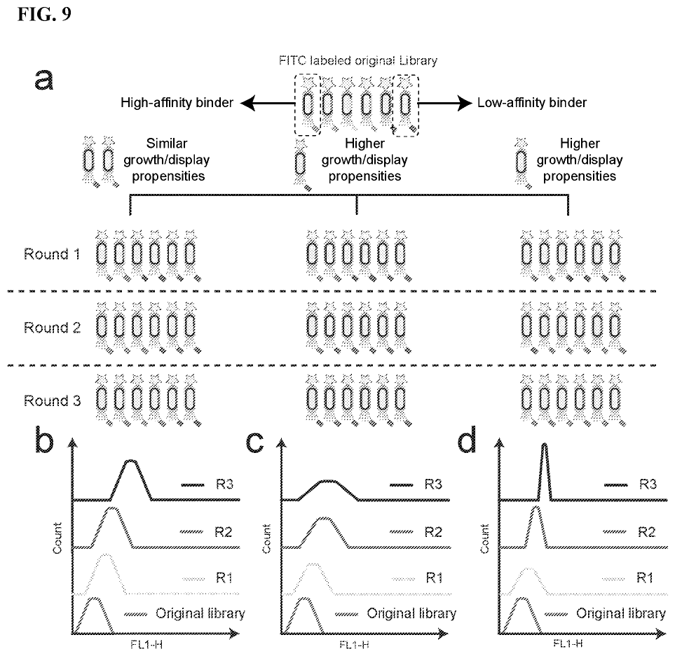

Similarly, by FITC labeling ph-Fab libraries from biopanning campaigns and analyzing by flow cytometry, an accurate, and quantitative distribution of ph-Fab bound to Ag-beads can be observed which is not achievable by previous techniques (i.e., phage ELISA). Applying this to successive rounds of biopanning in a single campaign allows for the distribution of binding to be monitored over time (FIG. 9). With the normalized FITC MFI (to a control sample) which indicates the average affinity of bound ph-Fab population, and the coefficient of variation (CV) which represents the range of affinities of individual binders, dominant factors that influenced the enrichment process during the biopanning campaign can be determined (FIG. 9). From accurate biopanning profiling data, the round which is the most enriched for Ag-Ab binding can be directly identified and prioritized for screening with BIAS. However, for cases where significant global Ag-Ab enrichment is not observed, minor higher, or shouldering fluorescent populations containing greater numbers of rare high-affinity binders can still be sorted (FIG. 3). As is common in recombinant Ab discovery with YSD coupled with FACS¹², it is possible to perform iterative rounds of ph-Fab labeling followed by fluorescence activated sorting, to rescue the enrichment process from potentially severe growth/display propensities and shift the selective pressure back to Ab-Ag binding.

### Example 2: Biolayer Interferometry Antibody Screen (BIAS) for Discriminatory Screening of Candidate Binders

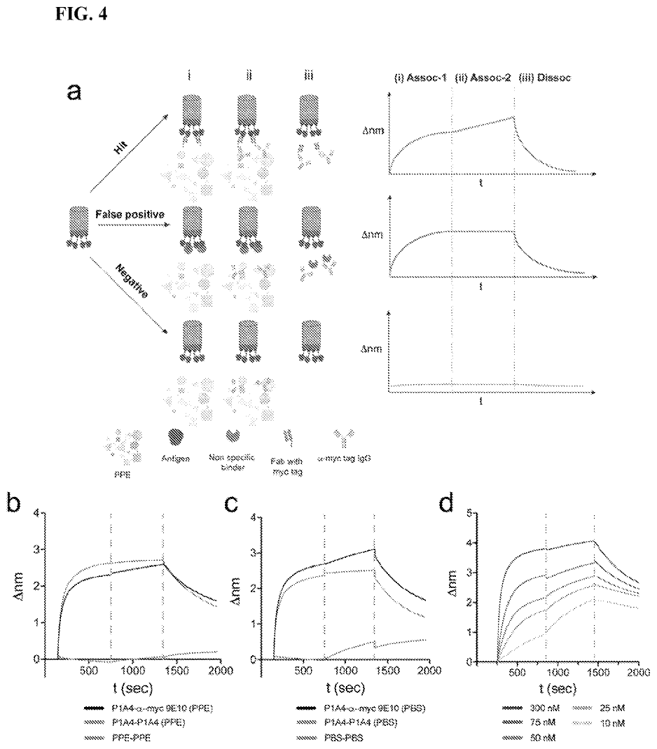

BIAS was developed for the discriminatory screening of candidate Abs by analyzing the real time binding of individual candidate Abs directly from crude periplasmic extract (PPE) samples using BLI. This is distinct from commonly utilized methods for candidate binder screening (i.e., dot blot, ELISA, DNA sequencing) where hits are not distinguishable based on their binding properties. BIAS consists of three steps (FIG. 4a). (i) Association-1 (Assoc-1): Ag loaded tips are transferred to crude PPEs containing expressed Fabs allowing Fabs to associate to the Ag, (ii) Association-2 (Assoc-2): tips are transferred to wells containing identical PPE+anti-tag IgG, where the anti-tag IgG can bind to the Fab (iii) Dissociation (Dissoc): tips (ii) are transferred to wells containing buffer where Fabs are dissociated. BIAS is able to identify true positive binders from a characteristic association curve observed in the Assoc-2 step which is absent for false positive clones. In the case that an observed association in Assoc-1 is occurring from a Fab, the secondary IgG will bind to its tag. A false positive signal in Assoc-1 arising from non-specific binding of periplasmic proteins will not show this second association signal as these non-specific proteins lack this tag. For our purposes, our entire ph-Fab library includes a myc-tag, therefore, we employed the anti-myc IgG, 9E10, for our Assoc-2 step. This Ab has been reported to be highly specific where one amino acid change has shown to disrupt binding 14 therefore binding in Assoc-2 is a direct readout of tagged Fab binding.

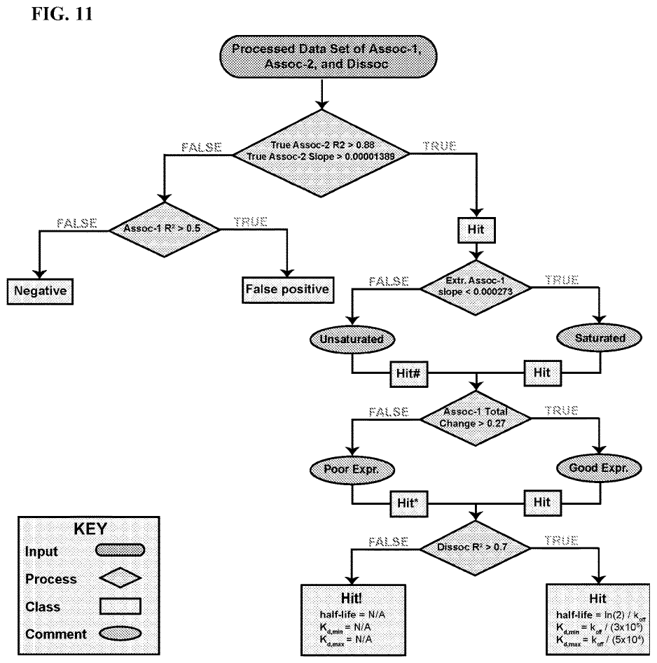

An in-house developed script, BIAS Algorithm Triaging Confirmed Hits (BATCH), individually analyzes the binding curves and categorizes each individual clone into “Hits”, “False positives”, and “Negatives” (FIG. 9). For “Hits”, we use predicted k_{off}s as the distinguishing factor for rank ordering binders as kon predictions are inaccurate due to the presence of inconsistent PPE in the Assoc-1 step (FIG. 11).

Characterizing BIAS with PIA4 and ARS1620 Labeled Peptide.

To establish proof of concept of the BIAS assay, we used the Fab-Ag pair, P1A4-ARS1620, (k_{off}=2.07×10⁻³/s, K_{d}=25.1 nM). P1A4 was tested against an ARS1620 labeled peptide at 250 nM (both Assoc-1 and Assoc-2) where the anti-myc IgG (9E10) was either included or absent in Assoc-2. Solutions of P1A4 spiked into both PBS and crude PPE derived from TG1 cells (E. coli) were tested in parallel to determine whether the method could tolerate the presence of the highly heterogenous PPE. An association curve is clearly observed in the Assoc-2 phase in samples that contains the secondary IgG, 9E10, when compared to the samples that do not (FIG. 4b, 4c) confirming that true positives are identifiable in the Assoc-2 step. Predicted values of kinetic parameters including k_{off}s from the BATCH yields near identical values to reported k_{off}s tested in vitro indicating that the addition of the Assoc-2 step or the PPE environment does not hinder kinetic predictions (table 1).

TABLE 1
BIAS with pure PIA4 spiked in PBS and PPE.

| | P1A4-9E10 (PBS) | P1A4-P1A4 (PBS) | P1A4-9E10 (PPE) | P1A4-P1A4 (PPE) |
|---|---|---|---|---|
| True Assoc-2 Slope (nm/s) | 7.1 × 10⁻⁴ | 1.33 × 10⁻⁴ | 3.94 × 10⁻⁴ | 1.39 × 10⁻⁴ |
| BIAS k_{off} (1/s) | 2.48 × 10⁻³ | 2.58 × 10⁻³ | 2.27 × 10⁻³ | 2.33 × 10⁻³ |

TABLE 2
BIAS kinetic parameters (k_{off}, truc Assoc-2 slope,
and extrapolated assoc-2 linear slope) of multiple
concentrations of P1A4 spiked in PPE.

| | | True | Extrapolated |
|---|---|---|---|
| | | Assoc-2 | Assoc-2 |
| P1A4 | BIAS | Slope | Linear slope |
| concentration | k_{off} (1/s) | (nm/s) | (nm/s) |
| 300 nM | 2.28 × 10⁻³ | 8.07 × 10⁻⁴ | 4.79 × 10⁻⁶ |
| 150 nM | 2.38 × 10⁻³ | 8.55 × 10⁻⁴ | 2.12 × 10⁻⁵ |
| 100 nM | 1.70 × 10⁻³ | 1.19 × 10⁻³ | 8.75 × 10⁻⁵ |
| 75 nM | 1.74 × 10⁻³ | 1.13 × 10⁻³ | 1.34 × 10⁻⁴ |
| 50 nM | 1.39 × 10⁻³ | 1.31 × 10⁻³ | 2.73 × 10⁻⁴ |
| 25 nM | 4.50 × 10⁻⁴ | 1.35 × 10⁻³ | 6.86 × 10⁻⁴ |
| 10 nM | 6.28 × 10⁻⁵ | 1.41 × 10⁻³ | 9.64 × 10⁻⁴ |

Varying concentrations of P1A4 (10⁻³⁰⁰ nM) were also tested to establish concentration dependencies of the assay (FIG. 4d). A series of concentrations of P1A4 spiked in PPE shows an increase in true Assoc-2 slopes as concentration approaches K_{d}. (table 2). Where the Assoc-1 step is not fully saturated, and the P1A4 sample concentrations of are at non-steady-state conditions, k_{off} predictions are less accurate. To address this, we developed BATCH to flag hits that show low expression or do not fully saturate in Assoc-1 to indicated inaccuracies in predicted k_{off} values for the user's discretion.

TABLE 3
BIAS hit summary table for CHIP.

| | Sample | Classi- | | Kd | |
|---|---|---|---|---|---|
| Rank | ID | fication | k off (1/s) | min (M) | Kd max (M) |
| 1 | F1 | Hit# | 9.40 × 10⁻⁶ | 3.13 × 10⁻¹¹ | 1.88 × 10⁻¹⁰ |
| 2 | F2 | Hit# | 8.17 × 10⁻⁴ | 2.72 × 10⁻⁹ | 1.63 × 10⁻⁸ |
| 3 | D11 | Hit# | 7.33 × 10⁻³ | 2.44 × 10⁻⁸ | 1.47 × 10⁻⁷ |
| 4 | C9 | Hit* | 1.47 × 10⁻² | 4.90 × 10⁻⁸ | 2.94 × 10⁻⁷ |
| 5 | D2 | Hit# | 1.87 × 10⁻² | 6.23 × 10⁻⁸ | 3.74 × 10⁻⁷ |
| 6 | A4 | Hit | 2.28 × 10⁻² | 7.61 × 10⁻⁸ | 4.57 × 10⁻⁷ |
| 7 | E4 | Hit! | 2.30 × 10⁻² | 7.67 × 10⁻⁸ | 4.60 × 10⁻⁷ |
| 8 | C5 | Hit | 2.60 × 10⁻² | 8.67 × 10⁻⁸ | 5.20 × 10⁻⁷ |
| 9 | G7 | Hit | 2.92 × 10⁻² | 9.75 × 10⁻⁸ | 5.85 × 10⁻⁷ |
| 10 | B3 | Hit! | 3.37 × 10⁻² | 1.12 × 10⁻⁷ | 6.74 × 10⁻⁷ |
| 11 | H11 | Hit | 4.50 × 10⁻² | 1.50 × 10⁻⁷ | 9.01 × 10⁻⁷ |
| 12 | G4 | Hit*! | 5.44 × 10⁻² | 1.81 × 10⁻⁷ | 1.09 × 10⁻⁶ |
| 13 | B10 | Hit*! | — | — | — |
| 14 | B11 | Hit! | — | — | — |
| 15 | D3 | Hit#! | — | — | — |
| 16 | D8 | Hit*! | — | — | — |
| 17 | E2 | Hit#! | — | — | — |
| 18 | F3 | Hit! | — | — | — |

Inaccurate k_{off} measurements are flagged (*-low expression #-unsaturuated Assoc-1, !- poor exponential fit for Dissoc). K_{d} min and max are estimated based on general kₒₙ of previously discovered Craik lab Fabs (5 × 10⁴-3 × 10⁵)

### RAPID Biopanning Enables the Identification of Rare Antibodies Against CH1P.

Carboxyl terminus of Hsp-70 interacting protein (CH1P) is an E3 ubiquitin ligase canonically known to interact with heat shock protein 70 (Hsp70) and heat shock protein 90 (Hsp90), leading to the ubiquitination of misfolded clients as well as regulation of chaperone turnover. Recent work demonstrated that CH1P has substrate specificity extending beyond Hsp70/90 and predicted interactions suggest CH1P may have additional Hsp-independent roles in proteostasis and disease states¹⁵. To enable further biological studies, we sought to develop Abs against CH1P beyond the available substrate binding inhibitors^{15,16}. CH1P is a challenging biopanning target due to its homodimeric structure and conformational flexibility, and therefore we employed RAPID biopanning.

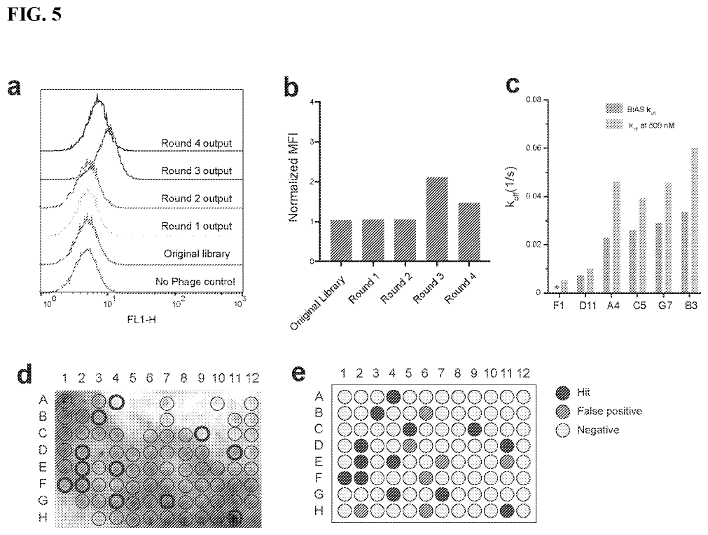

Four rounds of standard biopanning were employed against CH1P with increased stringency each round. Subsequently, RAPID flow cytometry was performed by individually FITC labeling each output round. Flow cytometry data shows an increase in normalized MFI from round 2 through 3, and a significant drop is observed at round 4 (FIG. 5a, 5b). The most enriched round (round 3) shows a normalized MFI of 2.11 which correlates to a reasonably high hit rate between 10 and 50 percent according to previous control experiments with P1A4. As flow cytometry data suggests strong Fab-Ag binding enrichment had occurred, fluorescent activated sorting was deemed unnecessary, and BIAS was performed directly.

Individual colonies were picked from round 3, and dot blots, ELISAs, and BIAS were performed using Fab expression induced crude PPEs. Using the parallel PPE samples, the dot blot resulted in 76 hits Out of 95 (80% hit rate) clones, while 18 hits were identified (19% hit rate) with BIAS (FIG. 5d, 5e, table 3). After exhausted optimizations, no hits were identified using ELISAs. Out of the 18 hits identified by BIAS, BATCH calculated k_{off}s showing a wide range (9.40×106-5.44×102). Four of the hits were labeled “Low expression” and 6 were flagged as “Unsaturated”. Six Fabs showed poor dissociation fits (R2<0.7), and therefore k_{off}s were not predicted. Candidate hit Fabs that were recombinantly expressed and biochemically characterized exhibit similar trends of kors compared to those predicted by BIAS (FIG. 5c). The biopanning campaign of CH1P demonstrates the power of RAPID biopanning as a method for accurately identifying the most enriched round, determining dominant factors of enrichment, and predicting hit rates of specific rounds of candidate hit screening. Notably, in this example, BIAS is shown to be a critical step in identifying true functional candidate hits and prioritizing potential binders for detailed biochemical characterizations.

TABLE 4
k_{off} (measured at 2 μM) and recombinant expression yields (E. coli) of Fabs from
CS3D biopanning.

| Fab | 3C8 | 3B11 | 3B12 | 2C11 | 3B12 | 4E4 | SP1-B3 |
|---|---|---|---|---|---|---|---|
| k_{off} (1/s) | 2.77 × 10⁻¹ | 2.05 × 10⁻¹ | 7.64 × 10⁻² | 4.19 × 10⁻¹ | 3.7 × 10⁻¹ | 1.11 × 10⁻¹ | 2.11 × 10⁻² |
| Expression | 2.7 | 2.4 | 1.7 | 0.9 | 1.4 | 0.3 | 0.1 |
| (mg/L culture) | | | | | | | |

### RAPID Biopanning Identifies Rare Binders Against CS3D

Cyclic STAT3 decoy (CS3D) is a dsDNA decoy that targets STAT3 and inhibits expression of downstream STAT3 induced genes^{17,18}. To date, only a few recombinant Abs have been developed against nucleic acid targets, with most of them targeting unique structural motifs^{5,6}. We present the identification of Fabs against CS3D, a 15-mer dsDNA target with no distinct structural motifs, as an example of a challenging target for which limited Ab discovery precedence exists. Both standard biopanning and RAPID biopanning were performed in parallel to provide a head-to-head comparison of the two methods.

First, standard biopanning (FIG. 1) was performed with Ag immobilized magnetic beads. A total of six rounds were performed with increasing stringency of washes and decreasing amounts of Ag per round. Assuming strong enrichment occurred continuously throughout the campaign, 95 colonies were randomly picked each round 4-6 (285 clones total) for candidate hit screening. Four unique Fabs (3B11, 3B12, 3C8, and 4E4) were identified from 38 randomly sequenced clones, where three of the Fabs (3B11, 3B12, and 3C8) differed by only one amino acid indicating that a strong enrichment had occurred for a specific family of related clones over others throughout the rounds. Purified Fabs exhibited weak binding against CS3D with k_{off} values ranging from 7.64 ×10⁻²-2.77 ×10⁻¹ at 2 IM Fab concentrations (table 4).

TABLE 5
BIAS of randomly chosen clones from Round 4-6 of
panning and Round 4 PhAB sorting.

| | | | | Round | Round 4 |
|---|---|---|---|---|---|
| | Round | Round | Round | 4 sorted | sorted |
| | 4 | 5 | 6 | (top 4%) | (bottom 96%) |
| Hits (#, *) | 69 | 84 | 77 | 37 | 69 |
| False | 17 | 11 | 17 | 106 | 21 |
| positive | | | | | |
| Negative | 9 | 0 | 1 | 47 | 5 |
| Total | 95 | 95 | 95 | 190 | 95 |

In efforts to identify improved binders, BIAS was performed with identical clones picked previously that were randomly sequenced. BIAS resulted in 230 hits out of 285 clones (81% hit rate). The top 10 ranked candidates were prioritized and sequenced, only to reveal the same low-affinity clones that were identified in the previous sequencing of random clones (Table 6), with the addition of two additional weak binders 2C11 (k_{off}, 4.19 ×10⁻¹) and 2B12 (k_{off}, 3.7 ×10⁻¹). The previously reported ARS1620 panning campaign resulted in much tighter binding Fabs (k_{off} range: 1.17 ×10⁻³-2.07 ×10⁻³) after four rounds. Therefore, we hypothesized that strong growth/display propensity biases were a dominant factor during the campaign, leading primarily to the identification of low-affinity binders.

TABLE 6
Top 10 BIAS ranked candidate clones and their k_{off}s at 2 μM.

| Round 4 | | | Round 5 | | | Round 6 | | |
|---|---|---|---|---|---|---|---|---|
| Clone | Rank | koff | Clone | Rank | koff | Clone | Rank | koff |
| 2A12, | 1, 3, 6 | 7.64 × 10⁻² | 3G3, 3F2, | 1, 2, 4, 5 | 2.05 × 10⁻¹ | 4G1, 4D12, | 1-10 | 7.64 × 10⁻² |
| 2C12, | | | 3C3, 3B11 | | | 4H1, 4C3, 4B1, | | |
| 2C9 | | | | | | 4F1, 4B2, 4C2, | | |
| | | | | | | 4E1, 4E2 | | |
| 2C6, 2A7 | 2, 8 | 2.05 × 10⁻¹ | 3F12, | 3, 6, 8, 10 | 7.64 × 10⁻² | | | |
| | | | 3C12, | | | | | |
| | | | 3E12, 3B12 | | | | | |
| 2B12, | 4, 5 | 3.70 × 10⁻¹ | 3C8, | 7, 9 | 2.77 × 10⁻¹ | | | |
| 2D6 | | | C9 | | | | | |
| 2B5, | 7, 9, 10 | 4.19 × 10⁻¹ | | | | | | |
| 2C11, | | | | | | | | |
| 2B2 | | | | | | | | |

Identical clones are italized or shown in bold.

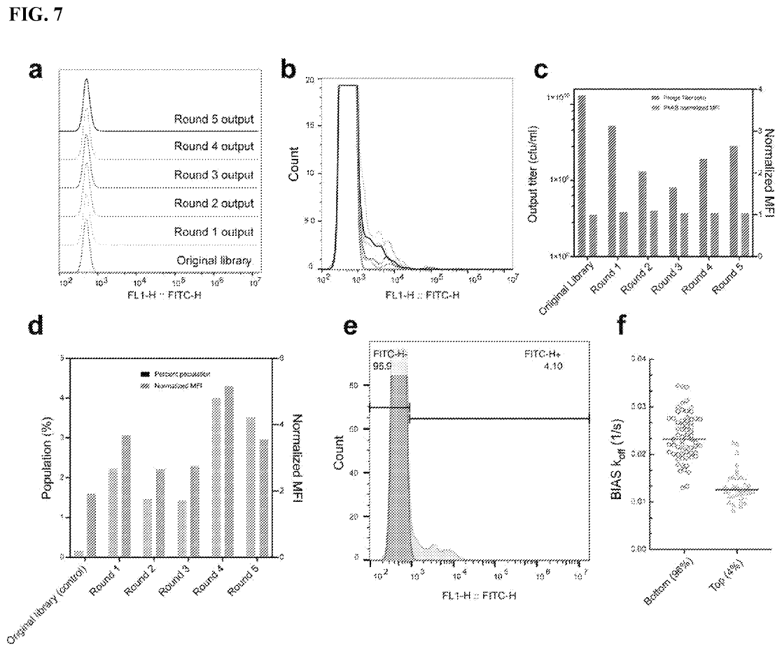

As continued efforts to identify higher affinity binders were unsuccessful using the standard method, RAPID biopanning was employed. First, the standard biopanning campaign enrichment profile was examined by FITC labeling output ph-Fab from each individual round of panning and subsequent analysis by flow cytometry (FIG. 7a, 7b). Results show that the normalized MFI exhibits no significant changes, despite titers of output phage showing enrichment in round 3-5 (534-fold increase in output titer from round 5 relative to that of round 3) (FIG. 7c). Interestingly, a minor shoulder distribution of higher fluorescent signal emerged as biopanning proceeds (FIG. 7b). Fluorescent activated sorting was employed to isolate this population.

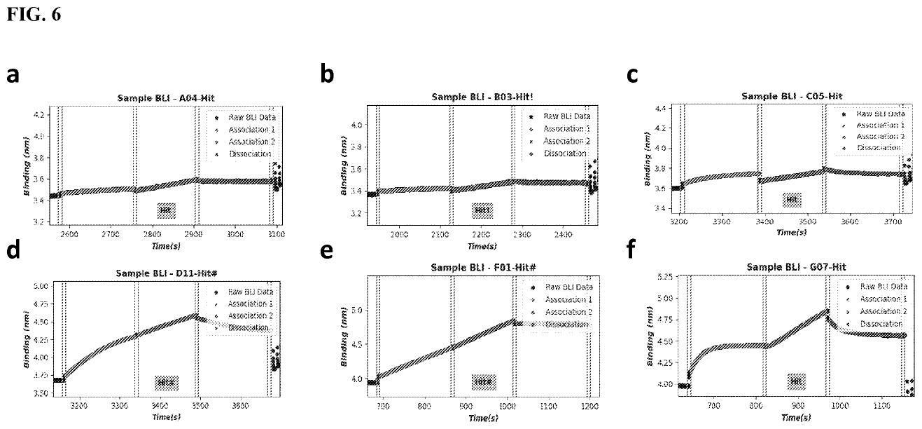

Fluorescent activated sorting was applied to round 4 as both the population percentage and the normalized MFI of the shoulder population are the highest (FIG. 7). The shoulder population (top ~4%) was sorted along with the majority population (bottom ~96%) and both populations were used to infect TG1 (E. coli) cells. BIAS was performed separately against both populations where ~2-fold lower average BIAS k_{off}s were observed in the top ~4% population hits compared to the bottom ~96% population hits (p value<0.001), suggesting the shoulder population contained ph-Fab that are more favorable (FIG. 6f). BIAS hits of the top ~4% sorted population were sequenced, and a new candidate binder, SP1-B3, was identified, along with identical clones of previously identified low-affinity binders. SP1-B3 exhibited 2.11 ×10⁻² (1/s) k_{off} which is ~3-fold lower than the previously identified Fab with the lowest k_{off}, and over ~20-fold lower than the Fab with the highest k_{off} (table 4). It is important to note that Fabs that were identified without RAPID biopanning exhibited significantly higher expression levels, as high as 27-fold (table 4). As it has been well reported that the expression levels of displayed protein correlate with display propensities¹⁹ it is reasonable to conclude that display propensity bias was a strong factor during the biopanning campaign of CS3D, which led to the enrichment of weakly binding but high expressing ph-Fabs. The use of RAPID in this panning campaign allowed the identification of the round most enriched for high-affinity binding Fabs and enabled the direct isolation of this population, resulting in the discovery of superior binding Abs.

### SUMMARY

The efficient identification of rare high-affinity Abs against challenging targets (i.e., low thermal stability, conformationally diverse, low expression of target, low antigenicity, low solubility, etc.) continues to be a major challenge in the Ab discovery field. For phage display based biopanning methods, challenging targets often exhibit weak Ab-Ag enrichment due to the low prevalence of high-affinity binders, and additional factors such as growth biases and inconsistent protein display propensities can affect, and even dominate the enrichment process. This ultimately leads to high-affinity binders never fully enriching and becoming difficult to identify, given the low probability of identification from common stochastic “hit-picking” screening methods.

The RAPID biopanning method was specifically designed as a solution for identifying rare high-affinity Abs against challenging targets using phage display. Where previous standard methods employ an approach where the total population of enriched displayed Abs are non-discriminatory screened, RAPID biopaninng isolates/identifies a selective population of high affinity binders that are subsequently screened in a discriminatory manner. This was achieved by (1) accurately identifying the most enriched population of ph-Fab, (2) increasing the prevalence of low frequency high-affinity ph-Fabs by fluorescent activated sorting, and (3) rapidly screening candidate hits in a discriminatory matter to prioritize in-depth biochemical characterizations of promising binders. We developed a simple method for fluorescent labeling ph-Fab for quantitative measurements of ph-Fab bounding to Ag immobilized beads and a novel BLI method, BIAS, was developed for rapid real time analysis of candidate binders. Ultimately, RAPID biopanning follows a Label-Profile-Sort-Screen pipeline (FIG. 1) and has been applied to two targets, CS3D and CH1P, where rare high-affinity binders were identified.

Robust labeling of ph-Fab was achieved using NHS-FITC. The labeling reaction is simple (only NHS-FITC and borate buffer required), fast (total reaction time 1 h in RT), reliable, and effective for the quantitative measurement of ph-Fab bound to Ag-Beads in flow cytometry. Most importantly, FITC labeling does not disrupt the binding of displayed Fab to Ag, nor does it exhibit any significant variabilities in labeling between different ph-Fabs (FIG. 2).

By individually FITC labeling ph-Fab libraries from a single biopanning campaign and analyzing bound ph-Fab on Ag-Beads on flow cytometry, the enrichment progression of the campaign can be profiled (FIG. 3a-3d). The progression of the global distribution of bound ph-Fab from subsequent rounds of biopanning can indicate dominant factors that affected enrichment (i.e., Fab-Ag binding, growth rates of phagemid containing E. coli, Fab display propensities). This is superior to phage ELISAs and calculations of phage titers of output ph-Fabs as these methods yield a single measurement of bound phage as opposed to a global distribution. This is highlighted in the CS3D biopanning campaign where output phage titers indicated enrichment from round 3-5, but flow cytometry data did not (FIG. 7a,-6c).

For campaigns that struggle to identify higher-affinity binders, fluorescent activated sorting of higher fluorescent populations can be an effective solution. As shown with the CS3D biopanning campaign, a rare higher affinity binder, SP1-B3, was identified by isolating the ph-Fab-Ag-bead complexes exhibiting higher fluorescent signal (top 4% shoulder population) by fluorescent activated sorting (FIG. 7e). It is worth noting that previous efforts of standard magnetic bead biopanning followed by random clone picking or even applying the more stringent BIAS screening to these clones failed to identify new higher-affinity binders, indicating that the pool of ph-Fab examined itself had severely low frequencies of higher-affinity ph-Fabs. Applying fluorescent activated sorting allowed for a selective population of higher-affinity ph-Fab to be isolated, thereby increasing its frequency and greatly improving the chances of isolating/identifying this attractive binder. In the case for the CS3D campaign, the significantly lower expression levels of SP1-B3 (as high as ~20-fold less) compared to the weaker binders suggests that, at least in part, Fab display propensities played a role in the enrichment process. The example of the CS3D biopanning campaign highlights the usage of fluorescent labeled ph-Fab libraries coupled with fluorescent activated sorting, where rare high-affinity binders can be retrieved from a large pool of weaker binders, previously enriched for factors other than Fab-Ag binding.

It is crucial to the outcome of the antibody discovery campaign to correctly prioritize candidate binders for in-depth biochemical characterization as time and resources are practically limited in a lab setting. To this point a discriminatory hit screening method, BIAS, was developed to use Abs from crude extracts to measure real-time binding to immobilized Ags using BLI. The raw data is analyzed with an in-house developed script, BATCH, and clones are categorized and rank ordered based on their kinetic off-rates. The discriminatory identification of clones allows for the prioritization of more promising candidates and further investigation and characterizations can be done in line with the BIAS rankings. As shown with the CH1P biopanning campaign, BIAS is more discriminatory compared to conventional screening methods such as dot blots and provides more in-depth details of candidate clones compared to ELISAs (i.e., Fab expression levels, k_{off} values etc. This is demonstrated by the most promising candidate binder, F1, showing no obvious indication of being a promising candidate from dot blot results (FIG. 5d, 5e). BIAS hits from the CH1P campaign show similar kinetic off-rate trends compared to the BIAS rankings, and of the five Fabs characterized, four show good agreement (~2-fold) of predicted k_{off} values compared to biochemically characterized k_{off}s.

RAPID biopanning, particularly where stringent fluorescent activated sorting was needed, can on occasion yield lower expression levels as shown with SP1-B3 (table 4) or be poorer growers. Expression of these binders in full-length IgG formats in mammalian cells can help overcome low expression yields in bacteria, which is a widely employed solution in improving Ab expression. As an example, SP1-B3 expressed in an IgG format shows a 220-fold increase in expression (33 mg/L culture) compared to its Fab counterpart.

While the RAPID biopanning pipeline synergizes high quality ph-Fab population isolation with rapid discriminatory screening of candidate clones, each step is highly modular and can be utilized independently or in combination with other existing protocols. For example, as is common with YSD coupled with FACS, iterative rounds of fluorescent activated sorting could be performed for continuous enrichment of high quality ph-Fab populations. Also, where functional assays are available for hit screening, functional screens can be utilized in place of BIAS, post flow cytometry profiling, and/or fluorescent activated sorting. Finally, BIAS can be utilized with different Ab formats (i.e., scFv, nonbodies etc.) and different anti-tag secondary IgGs as well.

In conclusion, it is demonstrated that RAPID biopanning couples efficiency with precision to allow for a more selective biopanning campaign to identify rare high-affinity candidate clones, particularly for challenging targets.

### Materials and Methods

### Standard Biopanning

Standard biopanning with magnetic beads were performed as previously described^{20,21}. Briefly, a human naïve B-cell phage displayed Fab library (diversity 4.1 ×1010) was used against CH1P and CS3D. Biotinylated Ags (EZ-link biotinylation Pierce) were immobilized to magnetic streptavidin beads (Dynabeads M⁻²⁷⁰ Streptavidin), and subsequently the ph-Fab library was added. For the CH1P campaign, 25 mM HEPES, 50 mM KCl was used for the binding, and 25 mM HEPES, 50 mM KCl, 0.05% Tween-20 was used for washing. For the CS3D campaign, 20 mM Tris-HCl, 150 mM NaCl, 0.05% NP-40, 1 mM EDTA was used for the binding, and both PBS and PBS-T (0.05% Tween-20) was used for washing. Both campaigns introduced negative selections against magnetic beads with no Ag starting at round 2. Individual clones were selected and screened with either Dot blots, ELISAs, and BIAS and subsequent hits were sequenced. Plasmids containing unique sequences of Fabs were used to transform BL21 (DE3) E. coli cells for further biochemical analysis of Fab.

### Phage Preparation

Fab-displayed phage were first amplified and prepared using standard methods. Briefly, 50 ml cultures (2×YT, 2% glucose, 100 μg/ml Ampicillin) of phagemid containing E. coli cells were incubated at 37° C. with 200 rpm shaking until OD600 reached ~0.5. Subsequently, 10 ml of this culture was infected with M13KO7 helper phage at 10:1 helper phage to cell ratio. Culture was incubated at 37° C. for 30 min without shaking followed by 20 min with shaking at 200 rpm. Infected cells were collected by centrifugation and cells were resuspended with fresh media (2×YT 100 mg/ml Ampicillin, 50 mg/ml Kanamycin). Cultures were grown overnight, and amplified phage were isolated by adding PEG 6000/2.5M NaCl phage to the supernatant of the overnighted culture. Phage yield was analyzed by taking OD268 measurements.

Phage Labeling with NHS-FITC

NHS-Fluorescein (Thermo Scientific) was prepared in DMSO (1 mg/ml final concentration). For the standard protocol labeling reaction consisted of the following: 50 μl of NHS-FITC (1 mg/ml) stock 500 μl of Phage (OD268=1, final), and 40 μl of Borate buffer (0.67M). Previous studies have shown that NHS-esters, including NHS-FITC, can efficiently label M13 phage via the N-termini and lysine residues of the pVIII coat proteins22,23. Labelling reaction was incubated in RT for 1 hr in the dark. PEG 6000/2.5M NaCl was added to the reaction to precipitate phage and incubated on ice for 15 min. Precipitated phage were collected by centrifugation (max speed for 5 min) and supernatant was removed. Pellets were resuspended in PBS and process was repeated. Phage were washed and precipitated three times total. Samples were immediately used in subsequent experiments. A plate reader (BioTek Synergy H4 plate reader) was used for fluorescent measurements and optical density of phage and FITC were analyzed using nanodrop. Phage labeling was aimed to maximize fluorescent signal per phage while maintaining solubility to achieve maximum dynamic range of fluorescent distribution. Labeling conditions were optimized using M13 helper phage. Normalized fluorescence shows an increase in relation to the number of FITC conjugated per phage, (FIG. 11a). Further labeling caused precipitation and was not further investigated. The washing and the reaction incubation time was also optimized (FIG. 11b,11c) where a final reaction time of 1 hour and a total of three washes determined to be optimal.

### Flow Cytometry and Fluorescent Activated Sorting

All experiments of flow cytometry biopanning profiling and fluorescent activated sorting were done with biotinylated Ags immobilized with streptavidin coated beads. SPHERO™ Streptavidin Polystyrene particles, (3.0-3.9 μm) were used to immobilize Ag. First, the beads were blocked with 2% BSA-buffer for 1 hour. Beads were then washed by resuspending beads in 1% BSA-buffer and subsequently centrifuging the beads (7k rpm for 2 min) to remove supernatant. This process was repeated three times. Biotinylated Ag was then added to beads at 1% BSA final concentration and incubated for one hour. Meanwhile, labeled phage were blocked in 1% BSA PBS for one hour. After Ag-immobilized beads were washed, blocked phage was added to the beads and incubated for 1-1.5 hours. After the binding phase, beads were washed 1-3 times with 1% BSA-buffer and passed through a 40 μm cell strainer. Subsequently, flow cytometry analysis or fluorescent activated sorting was performed. All sorting was performed on a BDFACS Aria II and all flow cytometry analysis were performed on a benchtop Beckman Cytoflex Analyzer or BDFACSCaliber machine. Bead populations were gated with FSC and SSC parameters and later only singlet populations were analyzed by gating linear FSA and FSW. Of singlet population of beads, histogram analysis was done with FITC. For fluorescent activated sorting, correct percentage of events were sorted. Sorted beads were used to infect fresh TG1 cells (OD600~0.7) and cells were plate for picking individual clones.

### 96-Well Periplasmic Extract Preparation

Single colony clones were picked and inoculated into 2×YT media containing 2% glucose and 100 mg/ml Ampicillin in round bottom 96 well plates (150 ml of media per well). Cultures were grown overnight at 37° C. with 200 rpm shaking. Following day 96 well cultures were inoculated (12 μl per well) into 96 well deep plates containing 2×YT media with 0.1% glucose and 100 μg/ml Ampicillin (1200 μl of media per well) and grown for 4-6 hours until culture is turbid. Fab expression was induced with 300 μl of 2×YT, ampicillin mg/ml and 5 mM IPTG and were left overnight at 30° C. with 200 rpm shaking. Periplasmic extracts were collected by osmotic shock. Briefly, cells from overnight cultures were collected by centrifugation at 2000 g for 25 min and 375 μl of ice-cold TES buffer (200 mM Tris-HCl, 500 mM EDTA, 500 mM Sucrose, pH=8) was added directly into each well and incubated with shaking at RT. Subsequently 1125 μl of ice-cold water was added in each well and mixed thoroughly. The periplasmic fraction (supernatant) was collected by centrifugation at 2000 g for 25 min and stored at −20° C. for future experiments.

### Dot Blots

From 96-well periplasmic extracts, 2-3 μl was applied to nitrocellulose membranes. After ~ 10 min, the membrane was blocked with 2%-TBS-milk and gently rocked at RT. After 1 hr, membrane was gently rinsed with TBS-T (0.05% Tween-20) and 1% TBS-Milk with anti-myc HRP (9E10) (1:5000 dilution) was added and incubated for 1 hr. Washes were performed with TBS-T (×2) and TBS (×2), and 1 ml of Immobilon Forte Western HRP substrate was added for imaging on a ChmiDoc MP imaging system.

### Biolayer Interferometry (BLI)

All buffers were filter sterilized with 0.22 mm filters prior to preparing samples. Black 384 well microplates were used to set up BLI plates and streptavidin tips were purchased by Sartorius. Kinetic constants of Fabs were determined by Octet RED384 system with continuous shaking at 1000 rpm in RT. Prior to the experiment, biological tips (model name) were presoaked in buffer to allow for equilibration for 1 hour. Data were analyzed using 1:1 interaction model on the ForteBio data analysis software 12.0.

### Biolayer Interferometry Antibody Screen (BIAS)

Instrument and reagent set up was identical to that of standard BLI. Anti-myc Ab (9E10) (Merk) was used for the Assoc-2. For the control experiments, BLI was run in following method: Baseline (1 min), Load, Baseline (1 min), Assoc-1 (10 min), Assoc-2 (10 min), Dissoc (10 min), and for CH1P and CS3D BIAS runs, Assoc-1 (3 min), Assoc-2 (2 min 30 sec), and Dissoc (3 min) was used as the final protocol. Dependent on the Ag that was used, loading step was adjusted where loading was immediately terminated once rate of loading changed to allow for equal distribution of Ag on tip. Raw data files of the run(s) were then used in the BATCH to rank order candidate hits according to their predicted dissociation rates. The true Assoc-2 slope of the PPE spiked sample without 9E10 calculated by BATCH was further used as the threshold value for determining hits (table 1).

### BIAS Algorithm Triaging Confirmed Hits (BATCH) Development

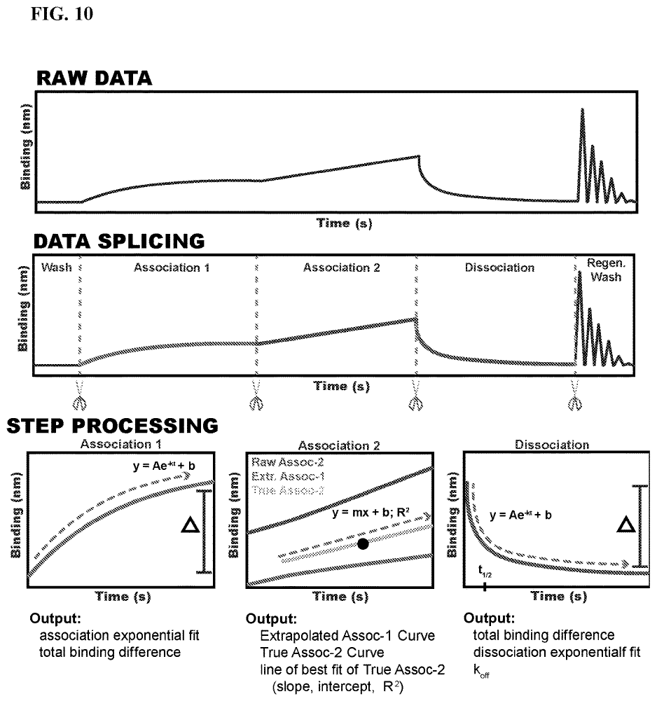

A BIAS function was developed to effectively process raw BLI sensogram data, to categorize and rank order candidate hits according to their kinetic properties. A brief schematic of the categorization, ranking system, and outputs is listed in FIG. 9. BATCH will process all raw BLI data files, a user-created methods file, and a user-created thresholds file. Due to buffer mismatches occurring during the BIAS runs, each step is trimmed at the beginning and end to avoid erroneous noise. Briefly, the Assoc-1 and Dissoc steps are fitted as a one phase exponential association and one phase exponential decay respectively. To account for continued Fab binding during the Assoc-2 step, the one phase exponential association fit of the Assoc-1 step is extrapolated through Assoc-2 as the “extrapolated association-1 curve”. The “true Assoc-2 slope” only accounts for the anti-myc IgG signal contribution and is calculated by subtracting the extrapolated association curve by the raw Assoc-2 curve. The “true Assoc-2 slope” is fitted with a linear regression to quantify a significant signal shift. Importantly the “true Assoc-2 slope” distinguishes “Hits” versus “False positives”. Threshold values were determined by control experiments (FIG. 4b-4d, table 1). BATCH outputs R2 values for all fits in each step which determines which category each clone is classified into based on a threshold value (FIG. 10). BATCH assumes a pseudo first-order kinetic model for predicting k_{off}s. By using the calculated k_{off}s from the Dissoc step, BATCH will predict a range of Kd based on previous Craik lab Fab kons and rank clones classified as “Hits”. BATCH will generate two tables to provide a summary of results and a more detailed view of all processed data. To give the user more information on the reason behind classification of clones, the comment section provides more details (i.e., no dissociation, no association, R2 values too low etc.). Threshold and kon values can be changed according to the user's experimental set up where secondary IgG or Ab format is different.

### Exceptions to the BIAS Rankings

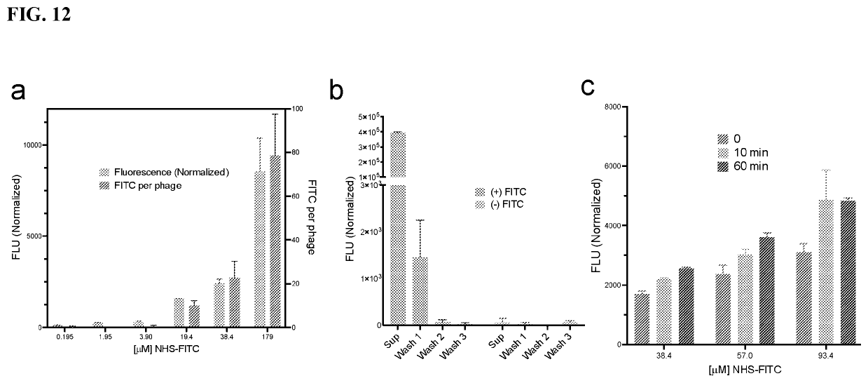

Clones that are flagged in the BIAS rankings indicate inaccuracies in k_{off} predictions. For these clones, the “true association-2 slope” can be a better predictor of binding, as exchange rates of Fab to Fab-9E10 complex during Assoc-2 are directly correlated to k_{off} of the Fab. Specifically, rapid exchange of Fab to Fab-9E10 indicate a high k_{off}, which translate to a high “true association-2 slope”. Therefore, k_{off} is inversely proportional to the true association-2 slope. A simple guide to further distinguishing flagged BIAS results is shown in FIG. 12.

### Fab Expression

Freshly transformed BL21 (DE3) E. coli single colony clones were picked and inoculated into 50 ml of 2×YT media containing 2% glucose and 100 mg/ml Ampicillin. Starter cultures were grown overnight at 370C with 200 rpm shaking. The next day, the starter culture was inoculated to 1 L of 2×YT media with 0.1% glucose and 100 mg/ml Ampicillin (OD600 0.05 final) and after incubation (37° C. with 200 rpm shaking), protein expression was induced with IPTG (1 mM final) at OD600 of 0.6 and continued to grow overnight (20° C. with 200 rpm shaking). Periplasmic extracts containing Fabs were collected by osmotic shock. Briefly, cells from overnight cultures were collected by centrifugation at 9000 g for 15 min and ~15 ml of ice-cold TES buffer was added and resuspended thoroughly to achieve a homogenous mixture. The mixture was then incubated with gentle shaking at 40C for 1 hour. Subsequently ~25 ml of ice-cold water was added and incubated with gentle shaking for 45 min. The periplasmic fraction (supernatant) was collected by centrifugation at 10,000 g for 30 min and loaded to Ni-NTA resin for affinity purification. Purified Fabs were dialyzed against PBS (10 kDa MWCO) and size exclusion chromatography was performed to further purify the Fabs and remove any aggregates (AKTA autopurification system with a Superdex 200 10/300GL column). Fractions exhibiting correct size were pooled and Fab concentrations were determined by absorbance at 280 nm.

Conversion of Fab into Full-Length IgG

The methods used here were described in detail in a previous study24. Briefly, the heavy and light chain regions of Fabs were cloned out from their respective phagemids and then individually cloned into a pTT5-SP-H1 mammalian expression vector through Gibson Assembly® (NEB #E2611) methods. The Gibson assembly product was transformed into NEB® 5× Competent E. Coli cells (NEB #C2987H) and plasmids were isolated using a ZymoPureII Plasmid Maxiprep Kit (Zymo Research #D4203) and confirmed through sequencing.

### Small-Scale Expression and Purification of Full-Length IgG in Mammalian Cells

The expression protocol used here was based on the Expi293™ Expression System (ThermoFisher #A14635). Expi293 suspension cells were seeded at a final density of 2.5×10⁶ cells/mL in a 6-well plate. The next day cells were diluted to a final density of 3×10⁶ cells/mL and a total of 1.0 μg/mL of plasmid DNA was transfected using ExpiFectamine™ 293 Transfection Kit. Transfected cells were then incubated on an orbital shaker at 37° C. and 8% CO₂ overnight and ExpiFectamine 293 Transfection Enhancer 1 and 2 were added to the cells. Secreted IgG was harvested 6 days post-transfection and the supernatant was incubated with Pierce™ Protein G Plus Agarose (ThermoScientific #22851) resin at room temperature for 1 hour. After washing with 10 column volumes of PBS, the IgG was eluted with 2 column volumes of 100 mM glycine, pH 2.5 and neutralized by 1 M Tris, pH 8.5. The eluted fractions were determined by SDS-PAGE gel and fractions containing pure IgG were collected and dialyzed against PBS buffer.

### REFERENCES

1. Basu, K., Green, E. M., Cheng, Y. & Craik, C. S. Why recombinant antibodies-benefits and applications. Pharm. Biotechnol.·Chem. Biotechnol. 60, 153-158 (2019).
2. Asarnow, D. et al. Structural insight into SARS-COV-2 neutralizing antibodies and modulation of syncytia. Cell 184, 3192-3204.e16 (2021).
3. Taylor, P. C. et al. Neutralizing monoclonal antibodies for treatment of COVID-19. Nat. Rev. Immunol. 21, 382-393 (2021).
4. Miersch, S. et al. Ultrapotent and broad neutralization of SARS-COV-2 variants by modular, tetravalent, bi-paratopic antibodies. Cell Rep. 39, 110905 (2022).
5. Zeraati, M. et al. I-motif DNA structures are formed in the nuclei of human cells. Nat. Chem. 10, 631-637 (2018).
6. Ye, J.-D. et al. Synthetic antibodies for specific recognition and crystallization of structured RNA. Proc. Natl. Acad. Sci. U.S.A 105, 82-87 (2008).
7. Fellouse, F. A. et al. High-throughput Generation of Synthetic Antibodies from Highly Functional Minimalist Phage-displayed Libraries. J. Mol. Biol. 373, 924-940 (2007).
8. Jones, M. L. et al. Targeting membrane proteins for antibody discovery using phage display. Sci. Rep. 6, 26240 (2016).
9. Koide, A., Wojcik, J., Gilbreth, R. N., Hoey, R. J. & Koide, S. Teaching an old scaffold new tricks: monobodies constructed using alternative surfaces of the FN3 scaffold. J. Mol. Biol. 415, 393-405 (2012).
10. Wellner, A. et al. Rapid generation of potent antibodies by autonomous hypermutation in yeast. Nat. Chem. Biol. 17, 1057-1064 (2021).
11. Lopez Tyler et al. Functional selection of protease inhibitory antibodies. Proc. Natl. Acad. Sci. 116, 16314-16319 (2019).
12. Boder, E. T. & Wittrup, K. D. Yeast surface display for screening combinatorial polypeptide libraries. Nat. Biotechnol. 15, 553-557 (1997).
13. VanAntwerp, J. J. & Wittrup, K. D. Fine affinity discrimination by yeast surface display and flow cytometry. Biotechnol. Prog. 16, 31-37 (2000).
14. Hilpert, K. et al. Anti-c-myc antibody 9E10: epitope key positions and variability characterized using peptide spot synthesis on cellulose. Protein Eng. Des. Sel. 14, 803-806 (2001).
15. Ravalin, M. et al. Specificity for latent C termini links the E3 ubiquitin ligase CH1P to caspases. Nat. Chem. Biol. 15, 786-794 (2019).
16. Ng, S. et al. Discovery and Structure-Based Design of Macrocyclic Peptides Targeting STUB1. (2021) doi: 10.26434/chemrxiv-2021-1108b.
17. Sansone, P. & Bromberg, J. Targeting the interleukin-6/Jak/stat pathway in human malignancies. J. Clin. Oncol. Off. J. Am. Soc. Clin. Oncol. 30, 1005-1014 (2012).
18. Johnson, D. E., O'Keefe, R. A. & Grandis, J. R. Targeting the IL-6/JAK/STAT3 signalling axis in cancer. Nat. Rev. Clin. Oncol. 15, 234-248 (2018).
19. Scott, N. et al. Single-chain Fv phage display propensity exhibits strong positive correlation with overall expression levels. BMC Biotechnol. 8, 97 (2008).
20. Duriseti, S. et al. Antagonistic anti-urokinase plasminogen activator receptor (uPAR) antibodies significantly inhibit uPAR-mediated cellular signaling and migration. J. Biol. Chem. 285, 26878-26888 (2010).
21. Kim, J. M., Stroud, R. M. & Craik, C. S. Rapid identification of recombinant Fabs that bind to membrane proteins. Methods 55, 303-309 (2011).
22. Yacoby, I., Shamis, M., Bar, H., Shabat, D. & Benhar, I. Targeting antibacterial agents by using drug-carrying filamentous bacteriophages. Antimicrob. Agents Chemother. 50, 2087-2097 (2006).
23. Li, K. et al. Chemical Modification of M13 Bacteriophage and Its Application in Cancer Cell Imaging. Bioconjug. Chem. 21, 1369-1377 (2010).
24. Duriseti, S. et al. Antagonistic anti-urokinase plasminogen activator receptor (uPAR) antibodies significantly inhibit uPAR-mediated cellular signaling and migration. J. Biol. Chem. 285, 26878-26888 (2010).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

While the subject antibody, method, and composition have been particularly shown and described with references to preferred embodiments thereof, it will be understood by those skilled in the art that various changes in form and details may be made therein without departing from the scope of the invention encompassed by the appended claims.

What is claimed is:

1. An isolated antibody that specifically binds to a cyclic STAT3 double-stranded oligonucleotide decoy (CS3D).

2. An isolated antibody that specifically binds to a cyclic STAT3 double-stranded oligonucleotide decoy (CS3D) and competes for binding to the CS3D with
(i) a first antibody comprising:
a variable heavy (VH) chain comprising heavy chain complementarity determining regions 1-3 (HCDRs 1-3) of a VH chain comprising the sequence:
(SEQ ID NO: 1)
QVQLQESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKGL
RYSSGWGQGTMVTVSS;
and
a variable light (VL) chain comprising light chain complementarity determining regions 1-3 (LCDRs 1-3) of a VL chain comprising the sequence:
(SEQ ID NO: 2)
QPVLTQPPSVSKGLRQTATLTCSGNSNNVGNAGAVWLQQHQGHPPKLLSY
RNNNRPSGISERISASRSGNTASLTISGLQPEDEADYYCSAWDSSLRVQV
FGGGTKLTVL;
or
(ii) a second antibody comprising:
a VH chain comprising HCDRs 1-3 of a VH chain comprising the sequence:
(SEQ ID NO: 3)
QVTLQQSGPGLVKPSQTLSLTCAISGDSVSSNSAAWNWIRQSPSRGLEWL
GRTYYRSKWFTYYAASVKGRISINRDTSKNQFSLQLNSVTPDDTAVYFCA
RGSGGYIDHWGQGTLVTVSS;
and
a VL comprising LCDRs 1-3 of a VL chain comprising the sequence:
(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

3. An isolated antibody that specifically binds to a cyclic STAT3 double-stranded oligonucleotide decoy (CS3D) and comprises:
a variable heavy (VH) chain comprising heavy chain complementarity determining regions 1-3 (HCDRs 1-3) of a VH chain comprising the sequence:
(SEQ ID NO: 1)
QVQLQESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKGL
RYSSGWGQGTMVTVSS;
and
a variable light (VL) chain comprising light chain complementarity determining regions 1-3 (LCDRs 1-3) of a VL chain comprising the sequence:
(SEQ ID NO: 2)
QPVLTQPPSVSKGLRQTATLTCSGNSNNVGNAGAVWLQQHQGHPPKLLSY
RNNNRPSGISERISASRSGNTASLTISGLQPEDEADYYCSAWDSSLRVQV
FGGGTKLTVL.

4. An isolated antibody that specifically binds to a cyclic STAT3 double-stranded oligonucleotide decoy (CS3D) and comprises:
a variable heavy (VH) chain comprising heavy chain complementarity determining regions 1-3 (HCDRs 1-3) of a VH chain comprising the sequence:
(SEQ ID NO: 3)
QVTLQQSGPGLVKPSQTLSLTCAISGDSVSSNSAAWNWIRQSPSRGLEWL
GRTYYRSKWFTYYAASVKGRISINRDTSKNQFSLQLNSVTPDDTAVYFCA
RGSGGYIDHWGQGTLVTVSS;
and
a variable light (VL) chain comprising light chain complementarity determining regions 1-3 (LCDRs 1-3) of a VL chain comprising the sequence:
(SEQ ID NO: 4)
QAVLTQPSSLSASPGASASLTCTLRSGINVDSYRIYWYQQKPGSPPQYLL
RYKSDSDKQQGSGVPSRFSGSKDASANAGILLISGLQSEDEADYYCMIWH
SSAVVFGGGTKLTVL.

5. The isolated antibody of any one of claims 1-4, wherein the antibody comprises:
a VH chain comprising a HCDR1 comprising the amino acid sequence GFTFSSYAMS (SEQ ID NO: 5), a HCDR2 comprising the amino acid sequence SAISGSGGSTY (SEQ ID NO:6), and a HCDR3 comprising the amino acid sequence GLRYSSG (SEQ ID NO:7), and
wherein the VH chain comprises an amino acid sequence at least 85% identical, at least 90% identical, at least 95% identical, at least 96% identical, at least 97% identical, or at least 98% identical to the amino acid sequence of SEQ ID NO:1.

6. The isolated antibody of any one of claims 1-5, wherein the antibody comprises:
a VL chain comprising a LCDR1 comprising the amino acid sequence SGNSNNVGNAGAV (SEQ ID NO:8), a LCDR2 comprising the amino acid sequence NNNRPS (SEQ ID NO:9), and a LCDR3 comprising the amino acid sequence SAWDSSLRVQV (SEQ ID NO:10), and
wherein the VL chain comprises an amino acid sequence at least 85% identical, at least 90% identical, at least 95% identical, at least 96% identical, at least 97% identical, or at least 98% identical to the amino acid sequence of SEQ ID NO:2.

7. The isolated antibody of any one of claims 1-4, wherein the antibody comprises:
a VH chain comprising a HCDR1 comprising the amino acid sequence GDSVSSNSAAWN (SEQ ID NO: 11), a HCDR2 comprising the amino acid sequence GRTYYRSKWFTY (SEQ ID NO:12), and a HCDR3 comprising the amino acid sequence GSGGYIDH (SEQ ID NO:13), and
wherein the VH chain comprises an amino acid sequence at least 85% identical, at least 90% identical, at least 95% identical, at least 96% identical, at least 97% identical, or at least 98% identical to the amino acid sequence of SEQ ID NO:3.

8. The isolated antibody of claim 7, wherein the antibody comprises:
a VL chain comprising a LCDR1 comprising the amino acid sequence TLRSGINVDSYRIY (SEQ ID NO:14), a LCDR2 comprising the amino acid sequence DKQQGS (SEQ ID NO:15), and a LCDR3 comprising the amino acid sequence MIWHSSAVV (SEQ ID NO:16), and
wherein the VL chain comprises an amino acid sequence at least 85% identical, at least 90% identical, at least 95% identical, at least 96% identical, at least 97% identical, or at least 98% identical to the amino acid sequence of SEQ ID NO:4.

9. The isolated antibody of any one of claims 1-8, wherein the antibody is a single chain Fv (scFv), IgG, Fab, (Fab) 2, or (scFv) 2.

10. The isolated antibody of any one of claims 1-9, wherein the antibody is a human antibody or a humanized antibody.

11. The isolated antibody of any one of claims 1-10, wherein the antibody is labeled.

12. The isolated antibody of any one of claims 1-10, wherein the antibody is attached to a solid support.

13. An isolated nucleic acid comprising a nucleotide sequence encoding the VH chain and the VL chain of the antibody of any one of claims 1-10.

14. A vector comprising the isolated nucleic acid of claim 13.

15. A cell comprising the nucleic acid of claim 13 or the vector of claim 14.

16. A composition comprising a first nucleic acid comprising a nucleotide sequence encoding the VH chain and a second nucleic acid comprising a nucleotide sequence encoding the VL chain of the antibody of any one of claims 1-10.

17. A cell comprising the first and second nucleic acids of claim 61.

18. A composition comprising a first vector comprising a nucleotide sequence encoding the VH chain and a second vector comprising a nucleotide sequence encoding the VL chain of the antibody of any one of claims 1-10.

19. A cell comprising the first and second vectors of claim 18.

20. A method for detecting a cyclic STAT3 double-stranded oligonucleotide decoy (CS3D) in a sample, comprising:
contacting an antibody of any one of claims 1-10 with the sample,
detecting binding of the antibody to CS3D in the sample.

21. The method of claim 20, wherein the antibody is labeled.

22. The method of claim 21, wherein the label is a fluorescent label, a chemiluminescent label, a radiolabel, an enzyme, or a chromogenic label.

23. The method of any one of claims 20-22, wherein the sample is blood, serum, or plasma.

24. The method of any one of claims 20-23, wherein the antibody is immobilized on a substrate.

25. The method of any one of claims 20-24, further comprising determining a concetration of CS3D.

26. A method for detecting presence of detecting a cyclic STAT3 double-stranded oligonucleotide decoy (CS3D) in vivo in a subject, the method comprising:
administering the antibody of any one of claims 1-10 to the subject; and
detecting the antibody in the subject,
wherein the subject has been administered the CS3D.
